(19) United States
(12) Patent Application Publication
(10) Pub. No.: US 2010/0297564 A1
(43) Pub. Date: Nov. 25, 2010

(54) POLYMERIZABLE FLUORINE-CONTAINING MONOMER, FLUORINE-CONTAINING POLYMER AND METHOD OF FORMING RESIST PATTERN

(75) Inventors: Tsuneo Yamashita, Osaka (JP); Yosuke Kishikawa, Osaka (JP); Yoshito Tanaka, Osaka (JP); Masamichi Morita, Osaka (JP)

Correspondence Address: SUGHRUE MION, PLLC, 2100 PENNSYLVANIA AVENUE, N.W., SUITE 800, WASHINGTON, DC, 20037, US

(73) Assignee: DAIKIN INDUSTRIES, LTD., Osaka-shi, Osaka (JP)

(21) Appl. No.: 12/810,076

(22) PCT Filed: Dec. 22, 2008
(86) PCT No.: PCT/JP2008/073326

§ 371 Date: Jun. 22, 2010

(30) Foreign Application Priority Data
Jan. 11, 2008 (JP) 2008-004932

(51) Int. Cl. G03F 7/20; C07C 69/65; C08F 120/22

(52) U.S. Cl. 430/325; 560/219; 526/245

(57) ABSTRACT

A polymerizable fluorine-containing monomer and a fluorine-containing polymer which are suitable for a resist layer and a protective layer of a laminated resist for forming a fine pattern in production of semiconductor devices, and further are useful especially in immersion lithography using water as a liquid medium, and a method of forming a resist pattern are provided. The polymerizable fluorine-containing monomer is represented by the formula (1):

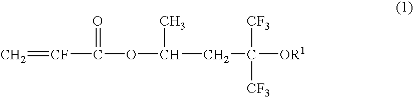

wherein R¹ is hydrogen atom or a monovalent saturated or unsaturated hydrocarbon group of 1 to 15 carbon atoms, and the hydrocarbon group may be chain or cyclic structure and may have oxygen atom, nitrogen atom, sulfur atom or halogen atom, the polymer is a homopolymer or copolymer of the monomer, and the method of forming a resist pattern using such a homopolymer or copolymer is carried out by immersion lithography.

### TECHNICAL FIELD

The present invention relates to a polymerizable fluorine-containing monomer, a fluorine-containing polymer and a method of forming a resist pattern, especially to a polymerizable fluorine-containing monomer and a fluorine-containing polymer which are suitable for a resist layer and a protective layer of a laminated resist for forming a fine pattern in production of semiconductor devices, and further are useful especially in immersion lithography using water as a liquid medium and to a method of forming a resist pattern.

Applications of the monomer and polymer of the present invention are not limited to the field of immersion lithography, and are used for various optical materials, for example, reflection preventing film, light-emitting element material, material for lens, material for optical devices, material for display, optical recording material, material for transmitting optical signal (optical transfer medium), and materials for sealing members thereof. In addition, the monomer and polymer of the present invention can be used as materials for medical use, for example, as coating materials for jointing portions and filters for various medical devices.

### BACKGROUND ART

Ultra-micro fabrication is needed for various electronic parts including semiconductor integrated circuit, and a resist is widely used in a technique for such fabrication. Also for multifunction and high density of electronic parts, there is an increasing demand for formation of an ultrafine resist pattern.

At present, in a photolithography technology for forming a resist pattern, a ArF lithography process in which exposing is carried out using ultraviolet light of a wavelength of 193 nm emitted from a ArF excimer laser is on the way to practical use as a leading edge technology.

In order to meet the requirements for forming further fine pattern for the coming generation, a F2 lithography process is under development in which exposing is carried out with ultraviolet light of further shorter wavelength of 157 nm emitted from a F2 laser, and on the other hand, there are proposed lithography technologies being applicable to further microfabrication using a ArF exposure system used in ArF lithography under development for practical use.

One of those technologies under investigation is an immersion exposure technology in a ArF exposure system in which a clearance between the reduction projection lens and the wafer having a resist film thereon is filled with pure water (“Immersion Optical Lithography at 193 nm” (Jul. 11, 2003) Future Fab Intl. Volume 15 by Bruce W. Smith, Rochester Institute of Technology).

Though light is passed in the air having a refractive index of 1 in a conventional process (dry method), when passing light in pure water having a refractive index of 1.44, in the case of the same incident angle of light for exposure, theoretically it is possible to obtain minimum resolution (minimum pattern line width) of 1/1.44.

The ArF exposing using those immersion exposing technologies is expected since a further fine pattern can be formed without greatly modifying various processes and equipment which have already been developed.

For example, a conventional ArF resist which is transparent at a wavelength of 193 nm, namely a resist material containing, as main component, a hydrocarbon resin having an aliphatic ring structure is under investigation.

Also, there are disclosed, as materials for a resist material which can be used for general resist pattern formation and as materials for a reflection preventing film, polymerizable monomers represented by:

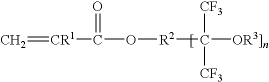

wherein R¹ represents hydrogen atom, halogen atom, a hydrocarbon group or a fluorine-containing alkyl group; R² is a straight-chain or branched alkyl group, a cyclic alkyl group, an aromatic group or a substituent thereof, and may be partially fluorinated; R³ is hydrogen atom, a hydrocarbon group which may have a branch, a fluorine-containing alkyl group, or a cyclic group having an aromatic or alicyclic structure, and may contain a bond of oxygen atom or carbonyl group; n represents an integer of 1 to 2, and polymers thereof (JP2003-40840A).

However, JP2003-40840A says that R¹ is halogen atom, but there is no description at all as to concrete examples of a monomer and polymer having R¹ of halogen atom and preparation process and characteristics thereof.

### DISCLOSURE OF INVENTION

At immersion exposing, since pure water is filled between the reduction projection lens and the resist film or protective layer, namely since the resist film or protective layer comes into contact with pure water, a static or dynamic water contact angle of the resist film or protective layer is desired to be large and the resist film or protective layer is required to be dissolved rapidly in a developing solution after the exposing.

The present invention has been completed as a result of intensive study in order to satisfy these conventional requirements.

Namely, the present invention relates to a polymerizable fluorine-containing monomer represented by the formula (1):

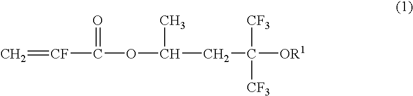

wherein R¹ is hydrogen atom or a monovalent saturated or unsaturated hydrocarbon group of 1 to 15 carbon atoms and the hydrocarbon group may be chain or cyclic structure and may have oxygen atom, nitrogen atom, sulfur atom or halogen atom.

The present invention also relates to a fluorine-containing polymer represented by the formula (I):

-(M)-(N)- (I)

wherein M is a structural unit derived from the polymerizable monomer represented by the formula (1); N is a structural unit derived from a monomer copolymerizable with the monomer represented by the formula (1), and the structural unit M is contained in an amount of 1 to 100% by mole and the structural unit N is contained in an amount of 0 to 99% by mole.

Further, the present invention relates to a method of forming a resist pattern by immersion lithography comprising:

(I) a step for forming a laminated resist for immersion lithography comprising a substrate and a photo-resist layer to be formed on the substrate,
(II) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(III) a step for treating the exposed laminated resist with a developing solution,
in which the photo-resist layer is characterized by comprising the polymer of the present invention, or
a method of forming a resist pattern by immersion lithography comprising:
(Ia) a step for forming a laminated resist for immersion lithography comprising a substrate, a photo-resist layer to be formed on the substrate and a protective layer to be formed on the photo-resist layer,
(IIa) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(IIIa) a step for treating the exposed laminated resist with a developing solution,
in which the photo-resist layer and/or protective layer is characterized by comprising the polymer of the present invention.

### BRIEF DESCRIPTION OF DRAWING

FIG. 1 A diagrammatic view for explaining each step (a) to (e) of the method of forming the first laminated resist and the method of forming a fine pattern by the immersion exposing of the present invention.

### EXPLANATION OF SYMBOLS

L0 Substrate
L1 Photo-resist layer
L2 Protective layer
11 Mask
12 Exposing region
13 Energy rays
14 Reduction projection lens
15 Pure water

### BEST MODE FOR CARRYING OUT THE INVENTION

The polymerizable fluorine-containing monomer of the present invention is the polymerizable fluorine-containing monomer represented by the formula (1):

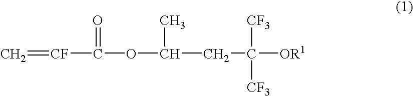

wherein R¹ is hydrogen atom, or a monovalent saturated or unsaturated hydrocarbon group of 1 to 15 carbon atoms and the hydrocarbon group may be chain or cyclic structure and may have oxygen atom, nitrogen atom, sulfur atom or halogen atom.

One of characteristics of this monomer is that a position of its acryloyl group is replaced by fluorine atom. By this α-fluoroacryloyl group, a speed of dissolution in a developing solution is remarkably increased as compared with a methacryloyl group and α-fluoroalkylacryloyl group which are described in JP2003-40840A.

R¹ is hydrogen atom, namely —OR¹ is OH, or a monovalent saturated or unsaturated hydrocarbon group of 1 to 15 carbon atoms and the hydrocarbon group may be chain or cyclic structure and may have oxygen atom, nitrogen atom, sulfur atom or halogen atom.

Examples of the monovalent saturated or unsaturated hydrocarbon group of chain structure are a straight-chain or branched alkyl group having 1 to 15 carbon atoms and a straight-chain or branched fluorine-containing alkyl group having 1 to 10 carbon atoms. These hydrocarbon groups may have oxygen atom, chlorine atom, bromine atom, iodine atom, carbonyl group, hydroxyl group, epoxy group, carboxyl group, amide group, cyano group, urethane group, amino group, nitro group, thiol group, sulfide group, sulfino group, sulfoxide group or sulfonic acid amide group in the chain or at the end thereof.

Examples of the straight-chain or branched alkyl group which has 1 to 15 carbon atoms and may have oxygen atom, nitrogen atom, sulfur atom or halogen atom (other than fluorine atom) are methyl, ethyl, propyl, isopropyl, butyl, t-butyl, pentyl,

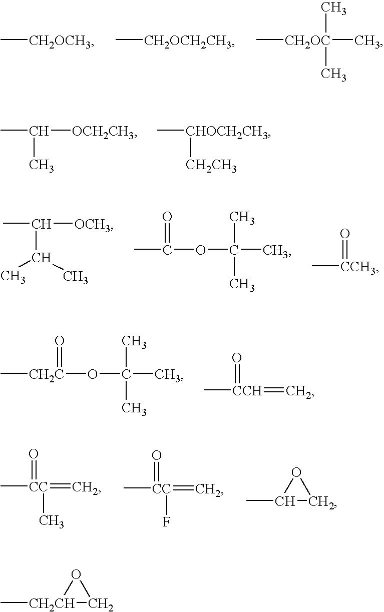

and the like. Among these,

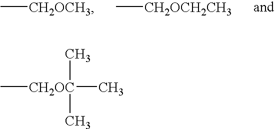

are preferred from the viewpoint of good deprotection reaction with a photo-acid generator, and

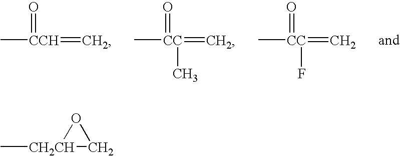

are preferred from the viewpoint of goof crosslinkability.

Examples of the straight-chain or branched fluorine-containing alkyl group which has 1 to 10 carbon atoms and may have oxygen atom, nitrogen atom or sulfur atom are

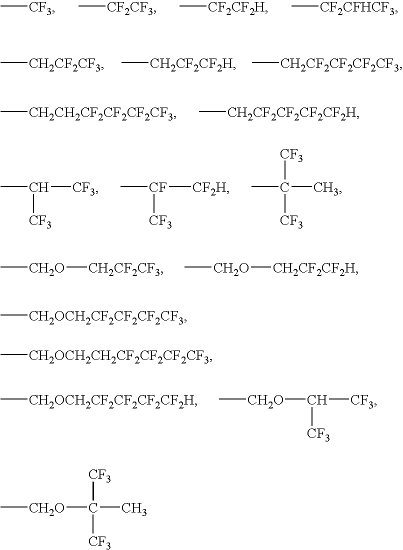

and the like, and among these,

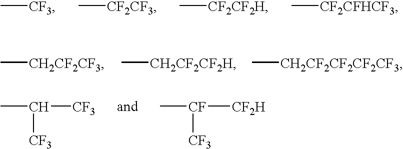

are preferred from the point that water repellency and liquid repellency can be improved without impairing solubility.

Examples of the monovalent hydrocarbon group of cyclic structure are hydrocarbon groups having 3 to 15 carbon atoms which have an aromatic ring structure or an aliphatic ring structure and may have fluorine atom, and these hydrocarbon groups may have oxygen atom, chlorine atom, bromine atom, iodine atom, carbonyl group, hydroxyl group, epoxy group, carboxyl group, amide group, cyano group, urethane group, amino group, nitro group, thiol group, sulfide group, sulfino group, sulfoxide group or sulfonic acid amide group in the chain or at the end thereof.

Examples thereof are

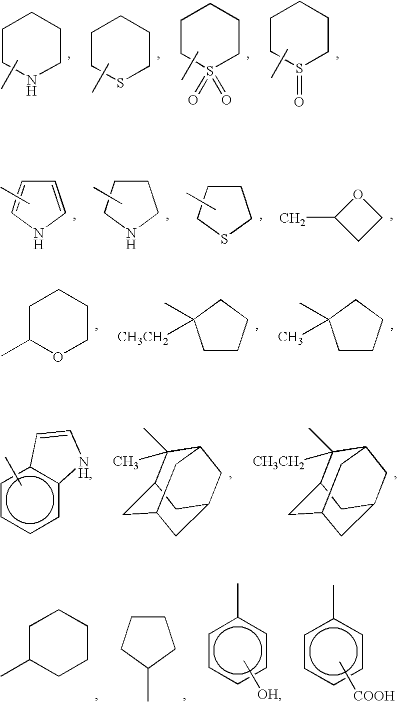

and the like. Among these, from the viewpoint of high transparency in vacuum ultraviolet region and good dry etch resistance,

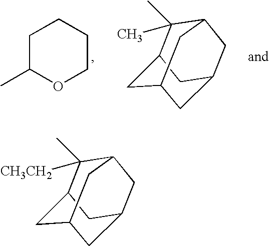

are preferred.

R¹ is especially preferably hydrogen atom from the viewpoint of good solubility in a developing solution.

The monomer represented by the formula (1) can be prepared, for example, by allowing α-fluoroacrylic acid (or α-fluoroacrylic acid fluoride or chloride, alkyl ester) to react with alcohol represented by the formula (3):

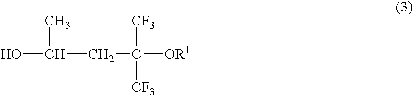

wherein R¹ is as defied in the formula (1).

With respect to the reaction conditions, those described in, for example, JP2003-40840A and Experimental Chemistry Course, Fifth edition, Vol. 16, pp. 35 to 38 and 42 to 43 can be employed.

The present invention also relates to a fluorine-containing polymer represented by the formula (I):

-(M)-(N)- (I)

wherein M is a structural unit derived from a polymerizable monomer (m) represented by the formula (1):

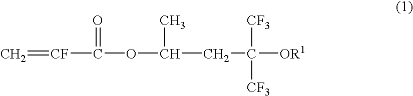

wherein R¹ is hydrogen atom or a monovalent saturated or unsaturated hydrocarbon group of 1 to 15 carbon atoms and the hydrocarbon group may be chain or cyclic structure and may have oxygen atom, nitrogen atom, sulfur atom or halogen atom; N is a structural unit derived from a monomer (n) being copolymerizable with the monomer represented by the formula (1), and the structural unit M is contained in an amount of 1 to 100% by mole and the structural unit N is contained in an amount of 0 to 99% by mole.

The polymer of the present invention may be a homopolymer of the polymerizable monomer (m) or a copolymer of the polymerizable monomer (m) and one or more of the copolymerizable monomer (n).

The above-mentioned monomer of the present invention is used as the polymerizable monomer (m).

The copolymerizable monomer (n) may be optionally selected depending on functions to be provided according to application and aimed object.

Examples of the material for the protective layer of the laminated resist for immersion lithography are monomers represented by the formula (22) in JP2007-204385A (R⁵ is H, CH₃, F or Cl), and among them, monomers represented by:

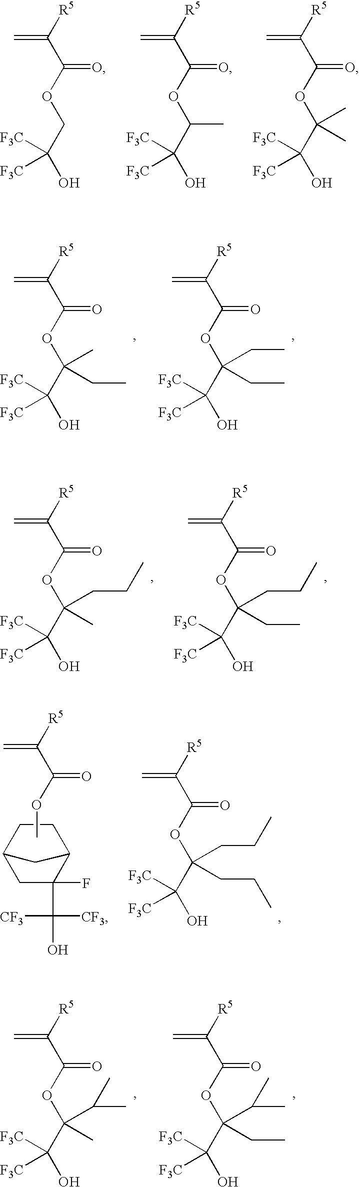

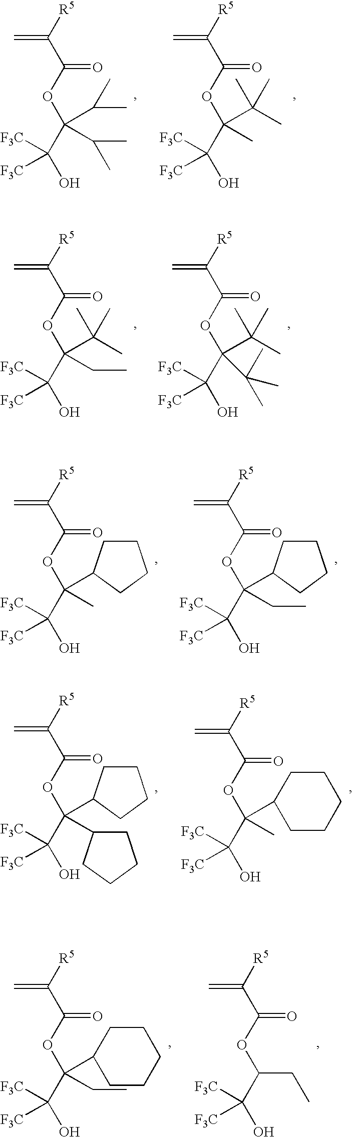

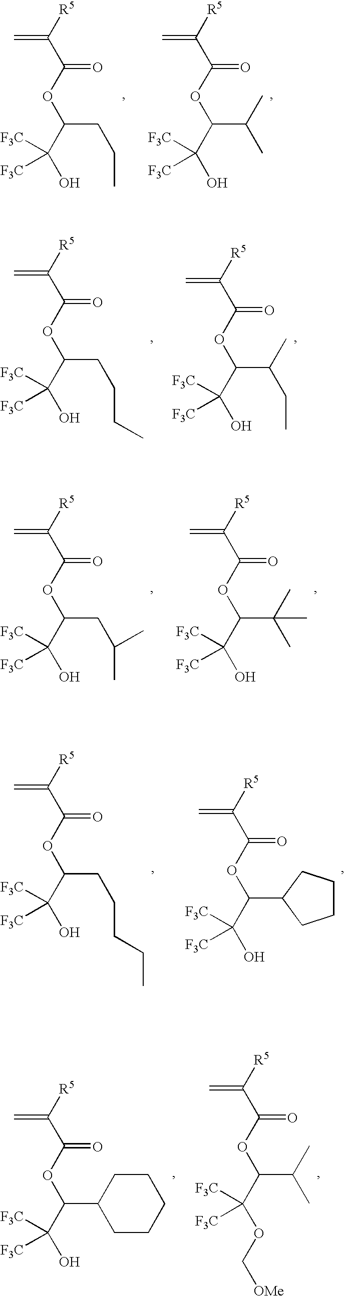

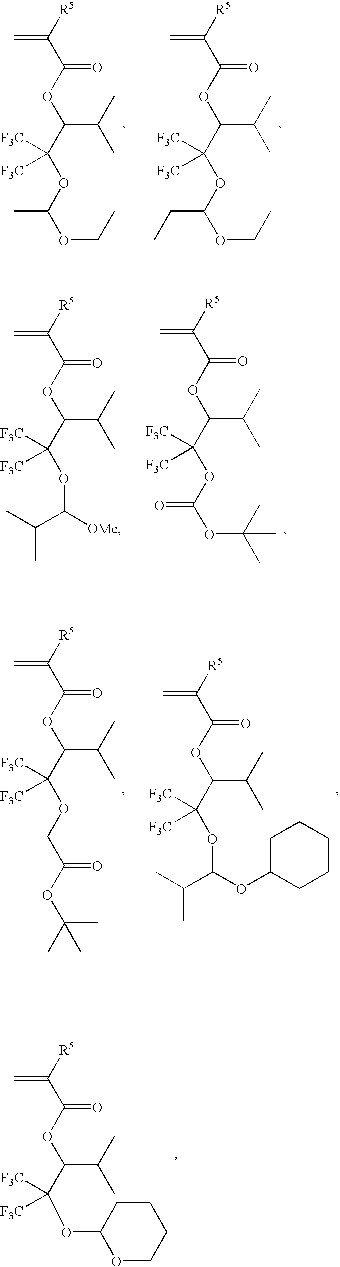

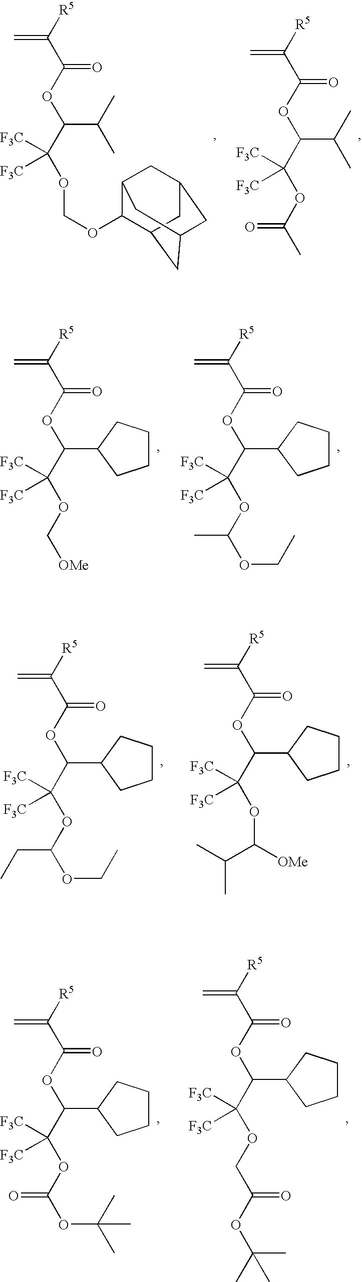

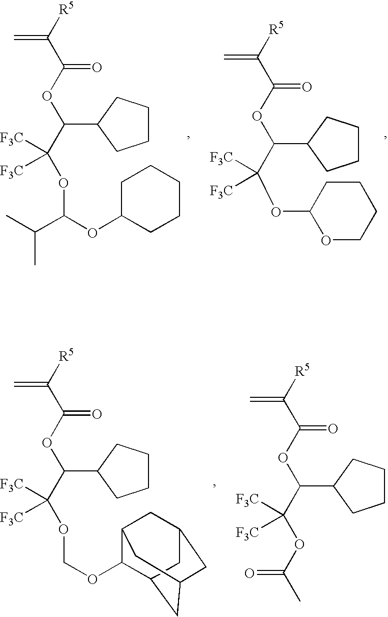

or monomers providing structural units represented by:

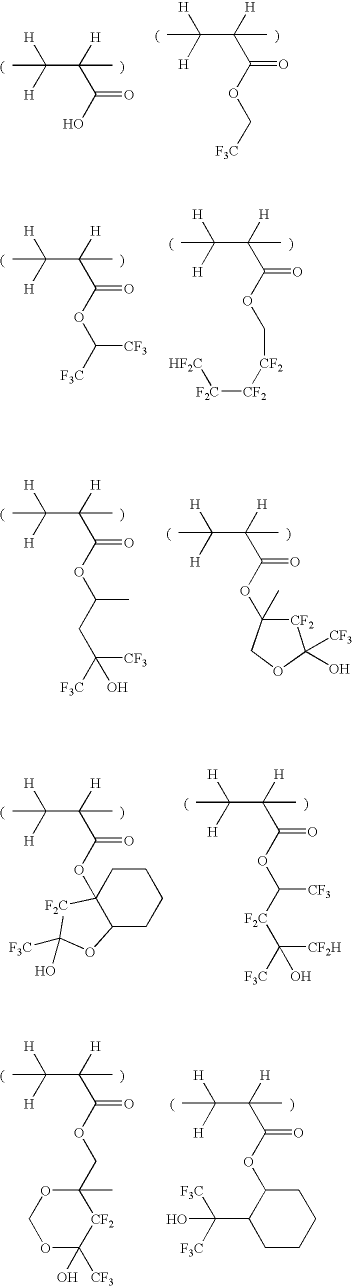

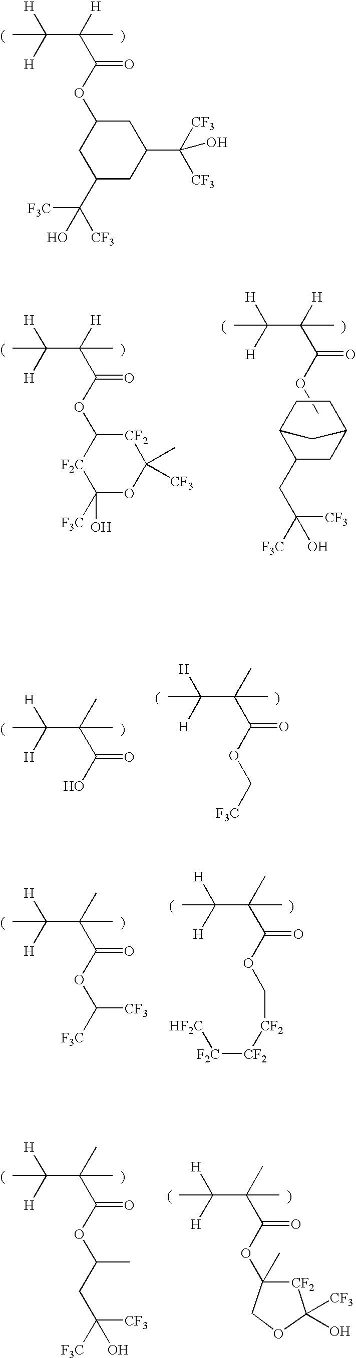

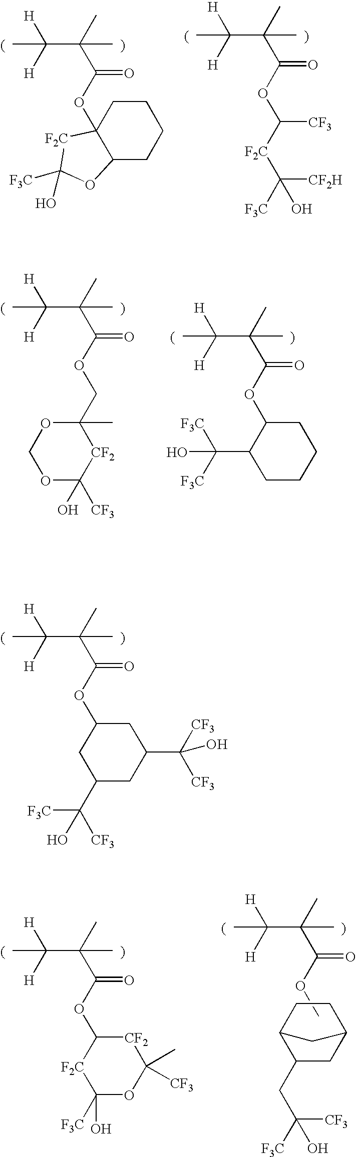

are preferred. In the present invention, especially preferred examples are:

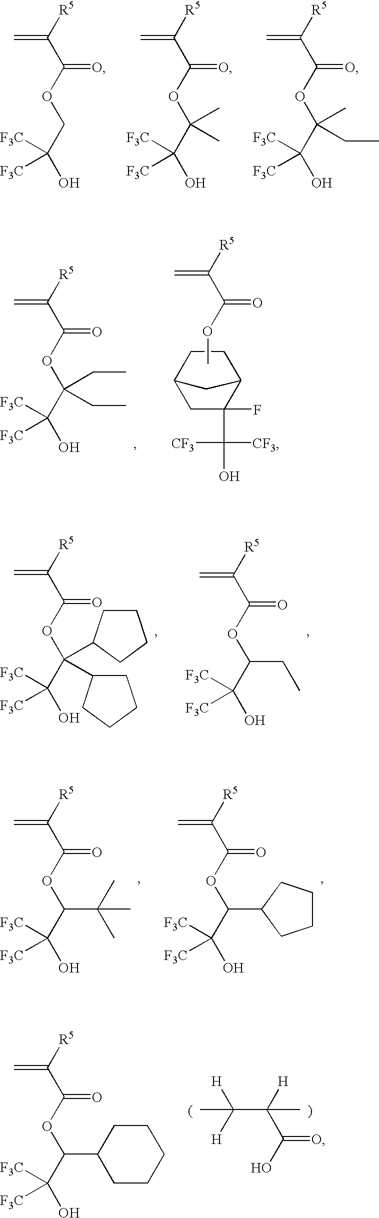

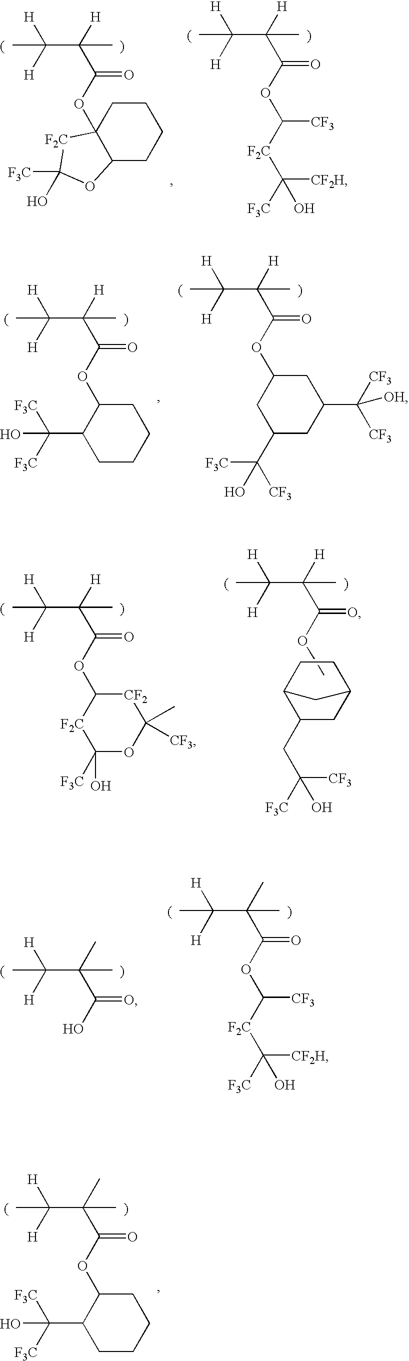

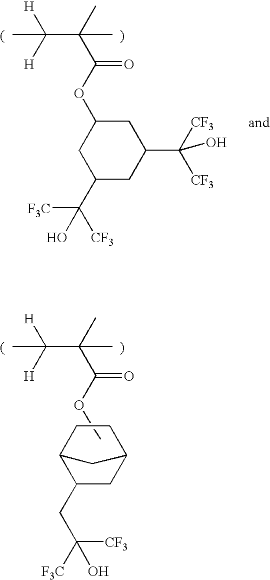

Further, compounds having F as R⁵ are preferred from the viewpoint of good polymerizability.

Examples of the material for the resist layer of the laminated resist for immersion lithography are monomers represented by the formulas (19) to (21) in JP2007-204385A (R⁵ is H, CH₃, F or Cl), and among them, monomers represented by:

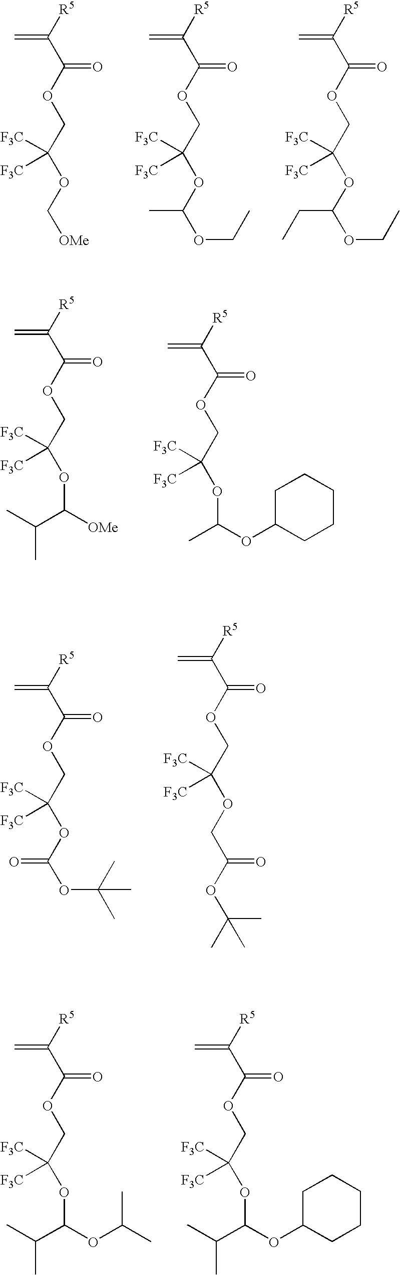

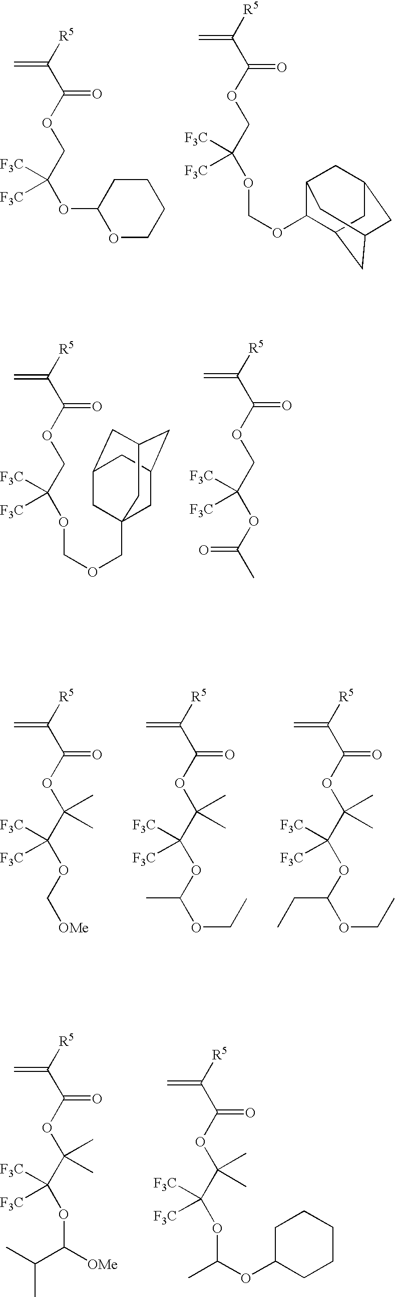

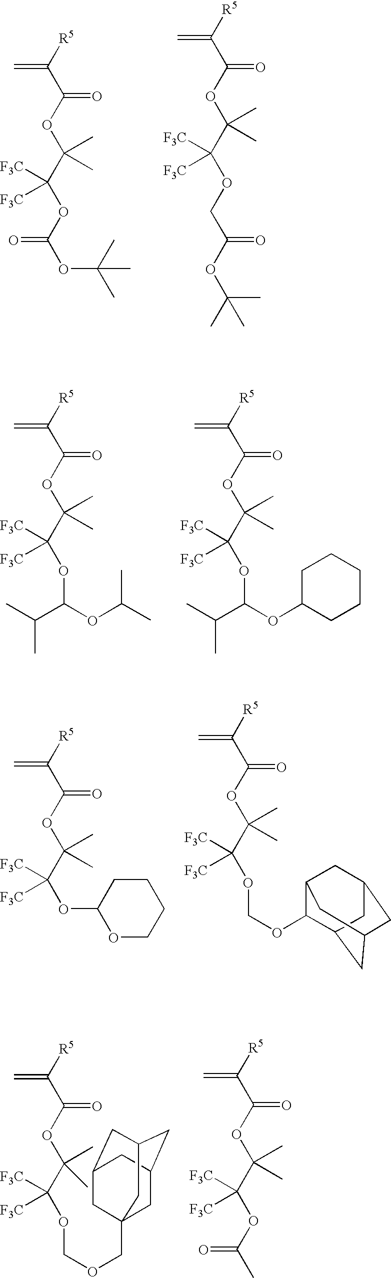

or monomers providing structural units represented by:

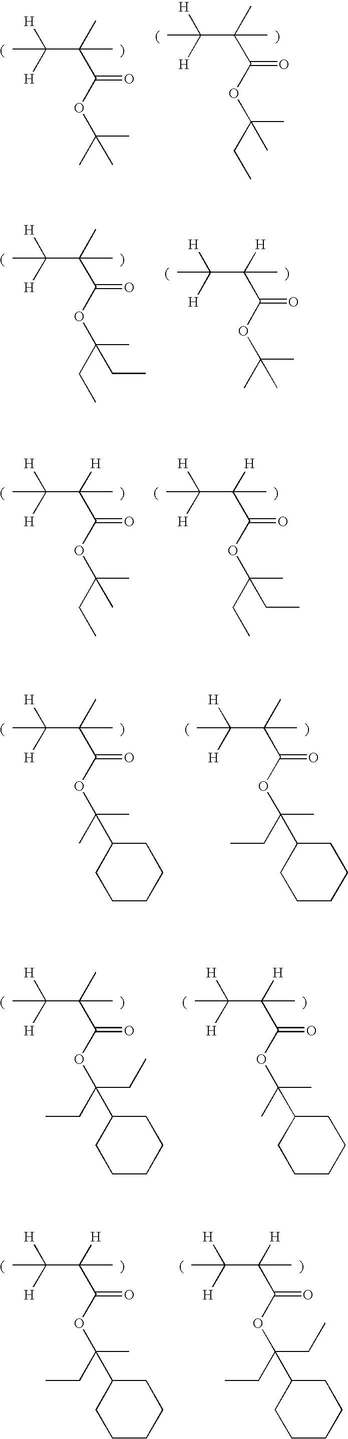

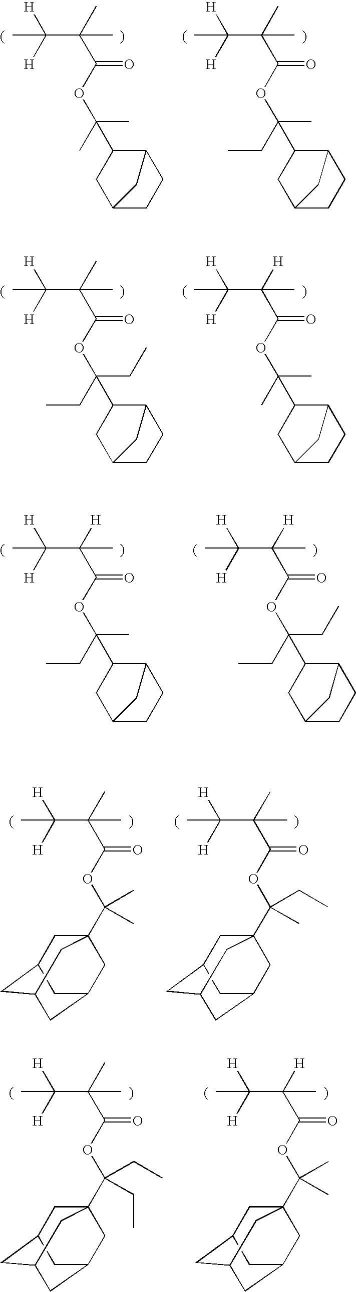

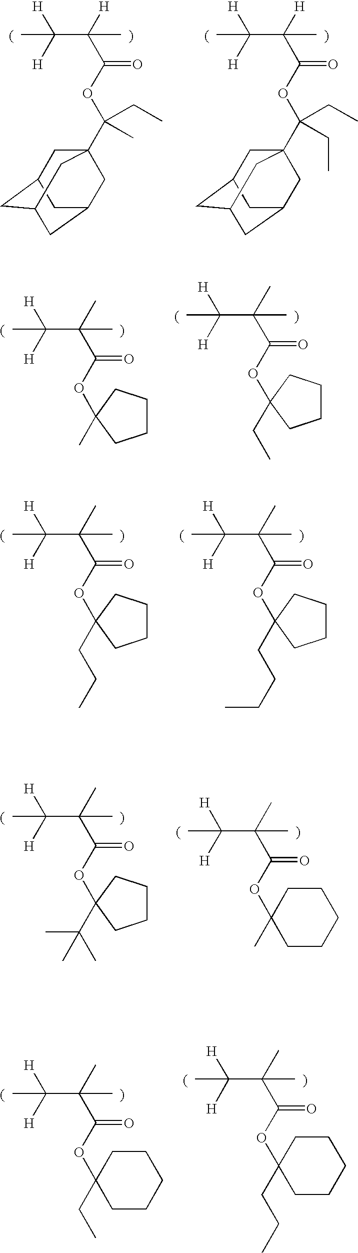

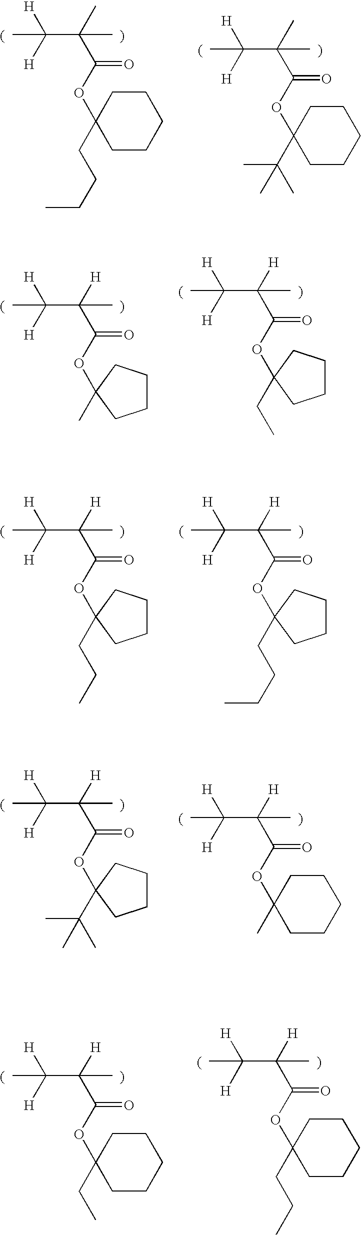

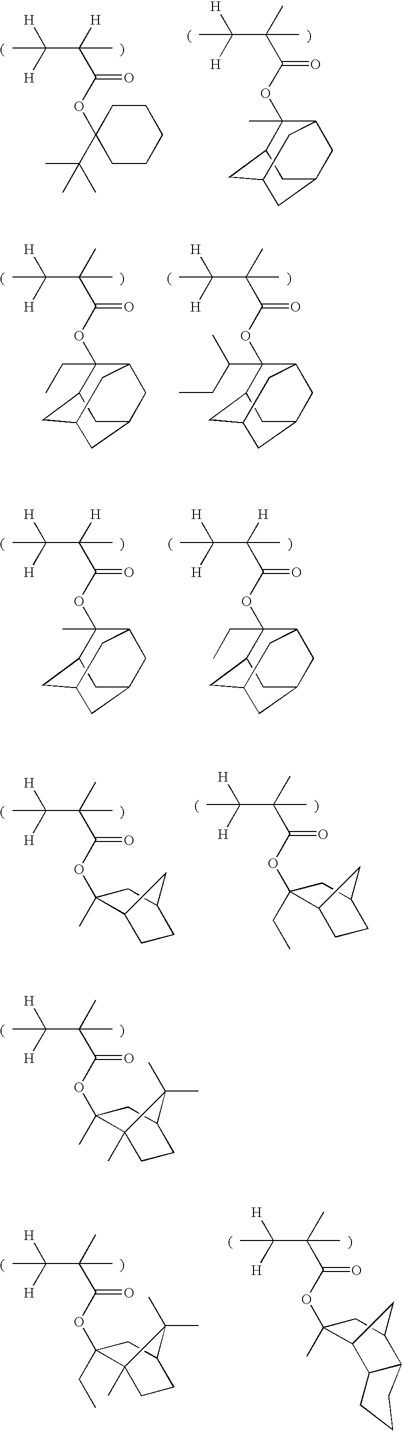

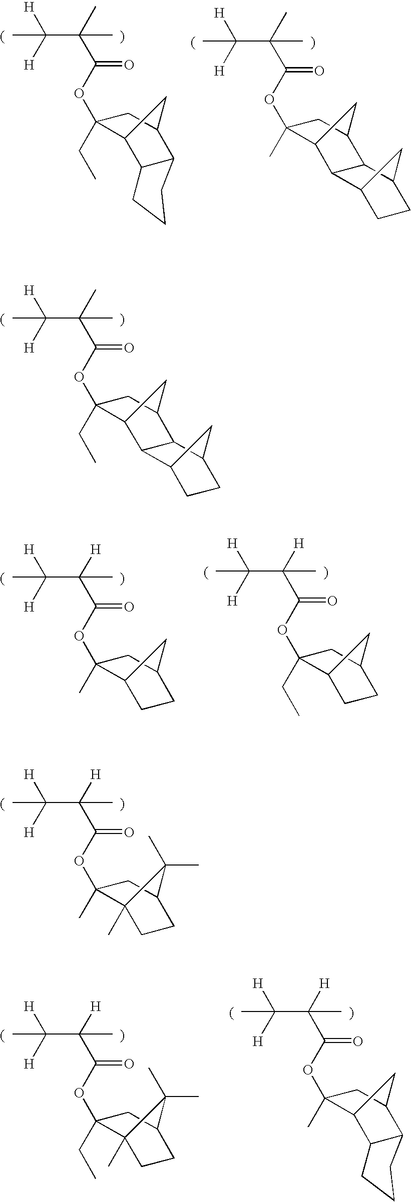

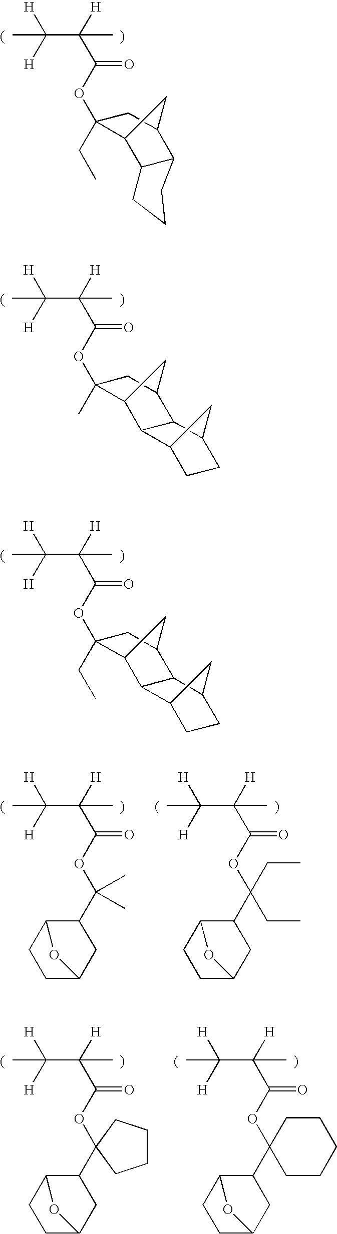

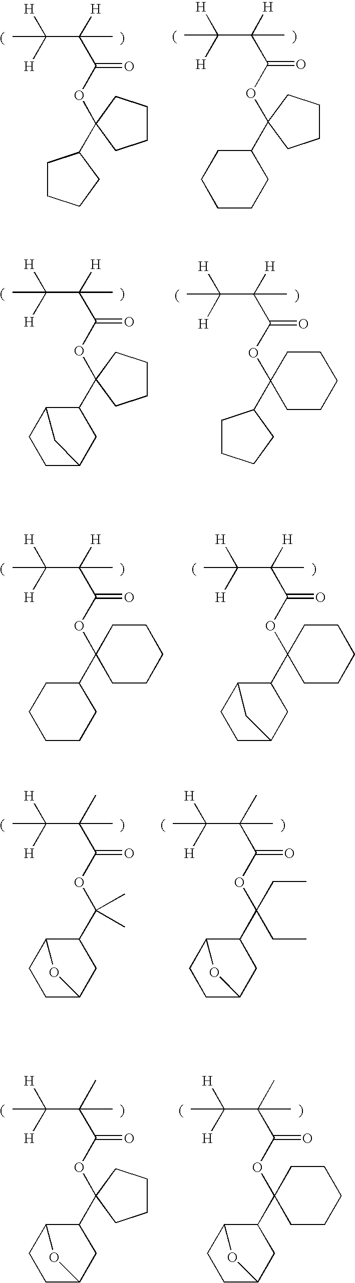

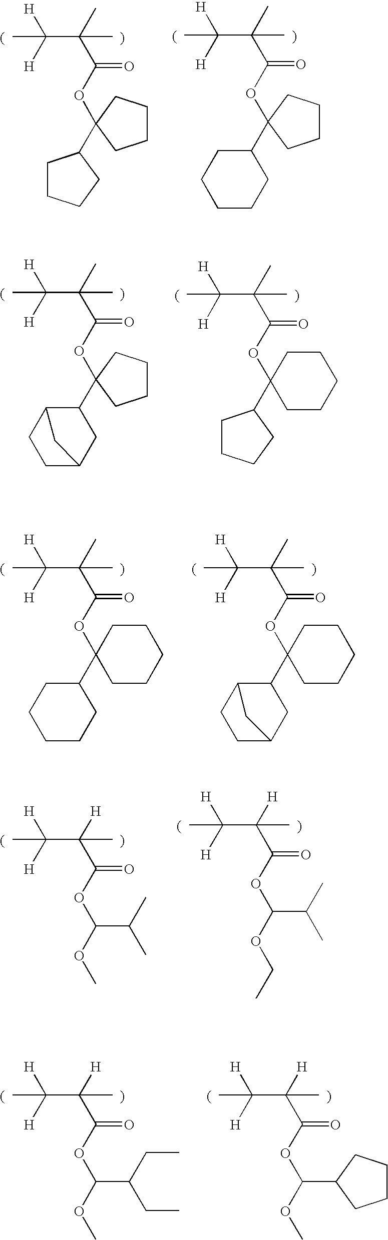

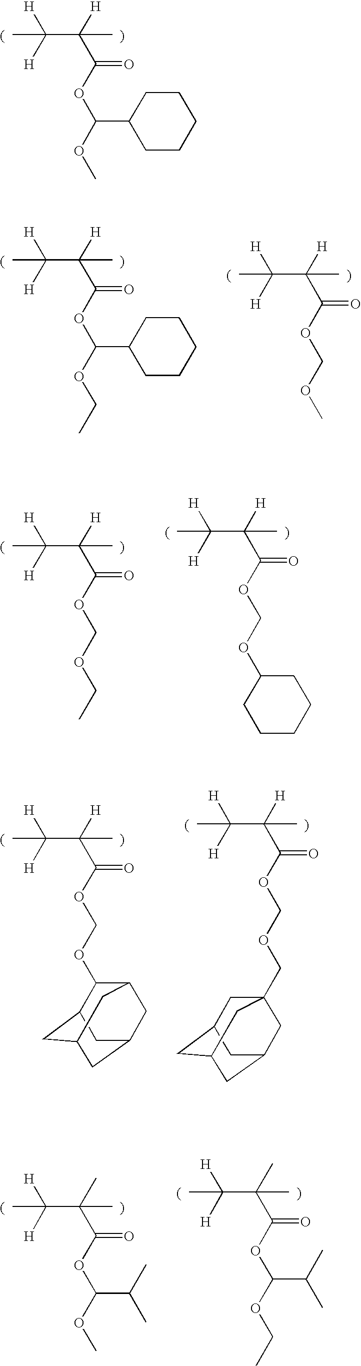

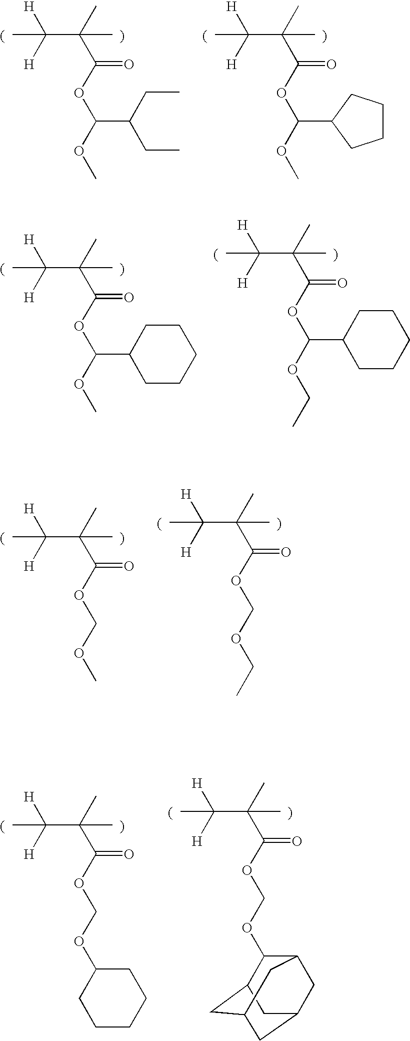

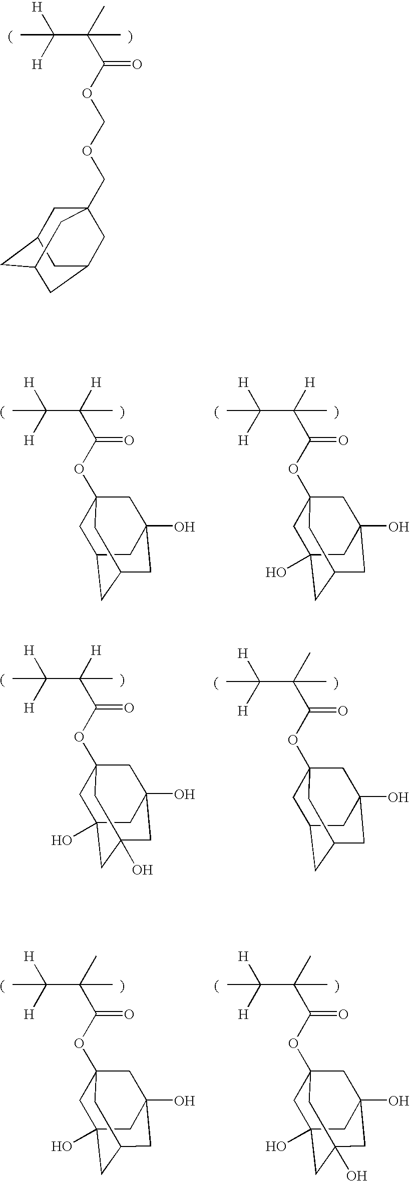

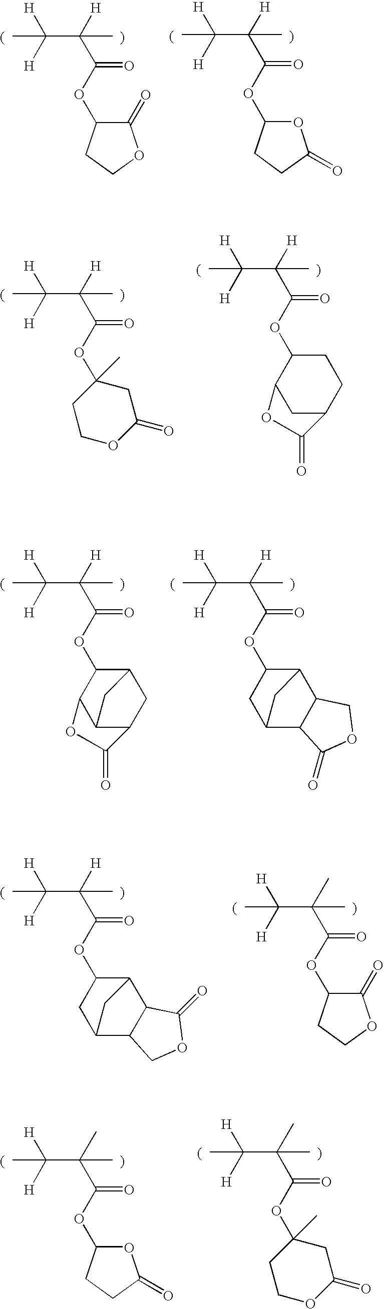

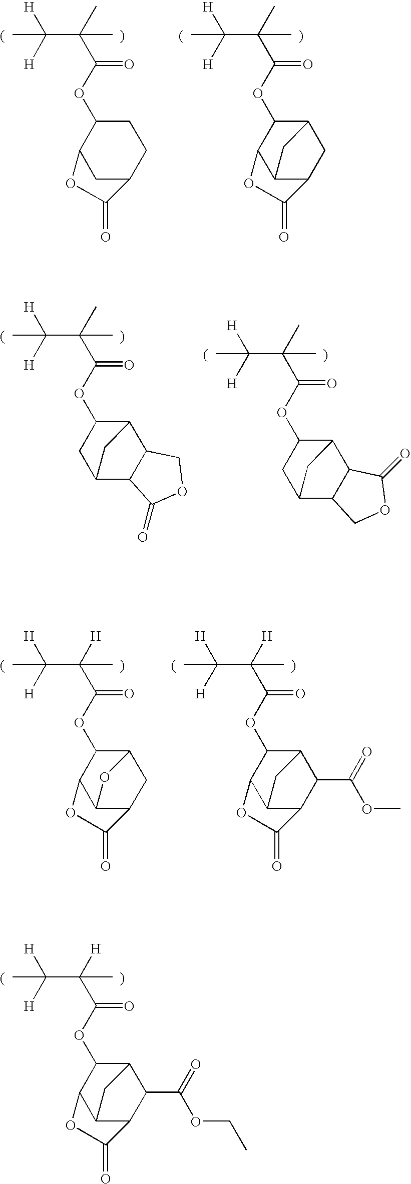

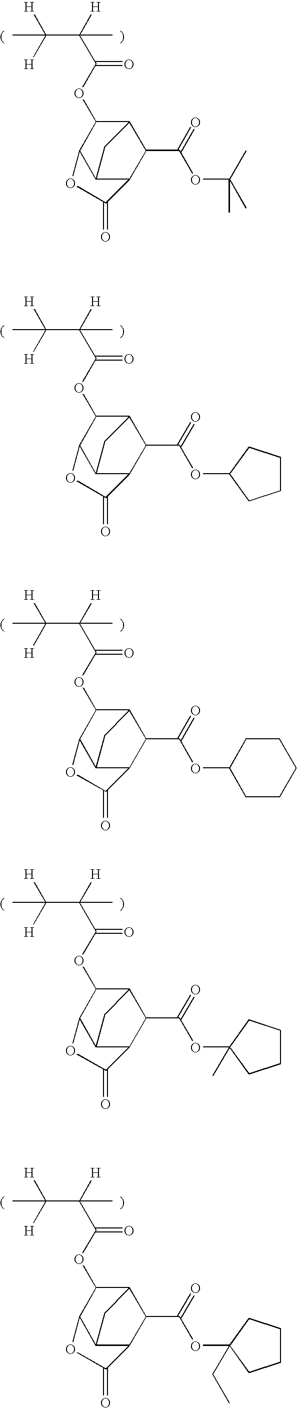

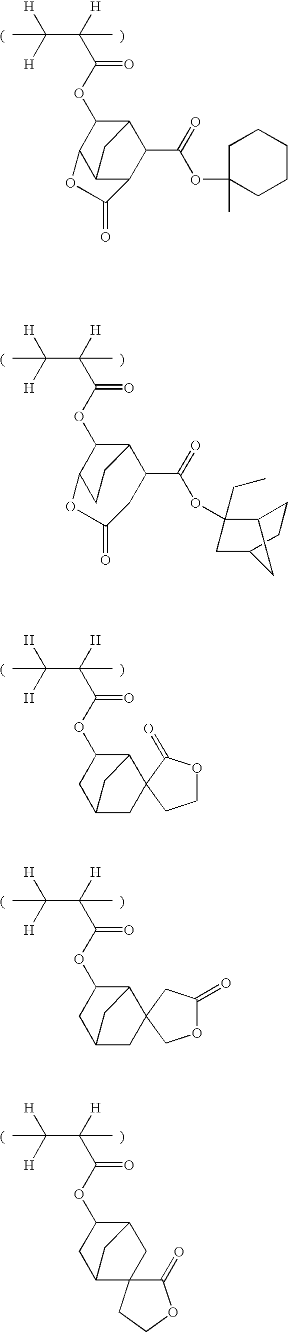

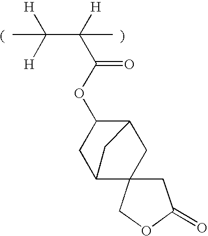

and examples thereof are preferred. In the present invention, especially preferred examples are:

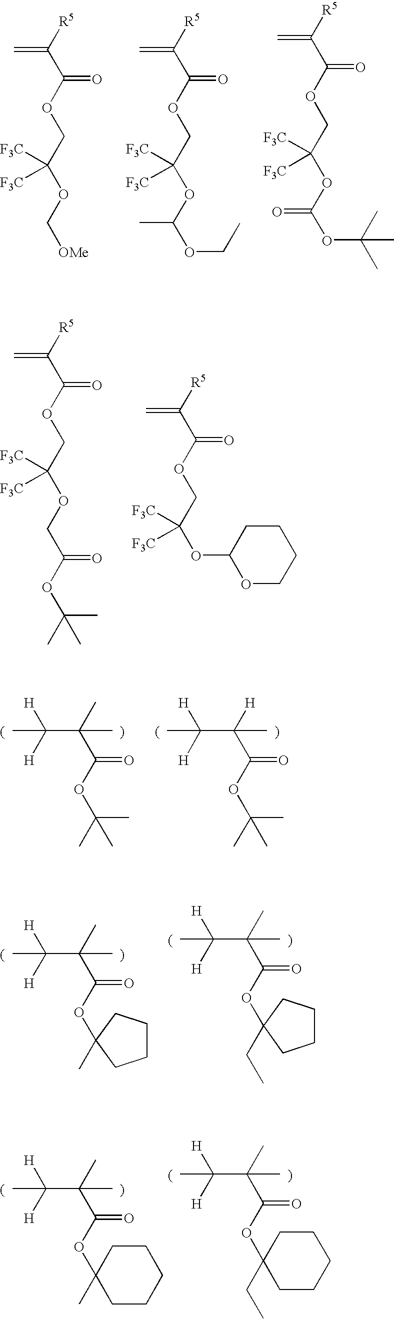

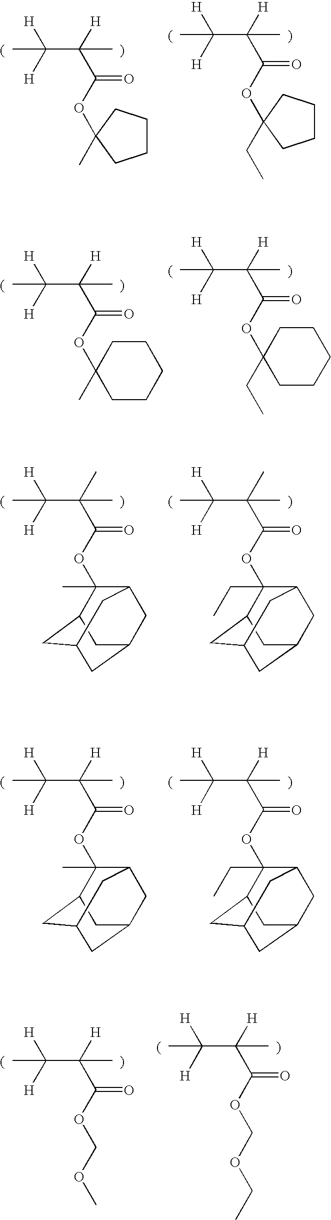

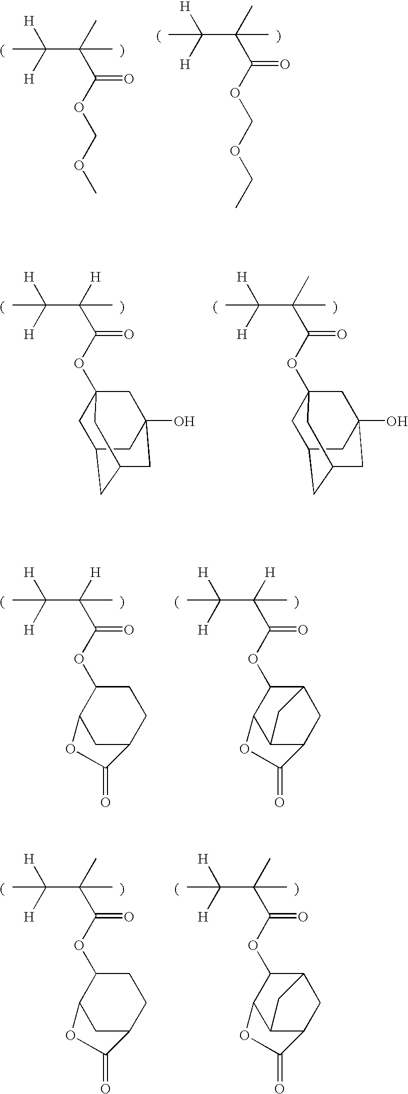

from the viewpoint of good dry etch resistance.

It is preferable that the structural unit M is contained in an amount of not less than 10% by mole, further not less than 30% by mole, from the viewpoint of maintaining good solubility. An upper limit of the structural unit N is 99% by mole.

A weight average molecular weight is preferably within a range from 1,000 to 1,000,000, and from the viewpoint of solubility, is preferably not more than 500,000, further preferably not more than 300,000, further preferably not more than 100,000. A lower limit is preferably 2,000, further preferably 4,000 from the viewpoint of film forming property.

The polymerization can be carried out by usual radical polymerization method, ionic polymerization method, iodine transfer polymerization method and metathesis polymerization method.

The polymer of the present invention is used as materials for laminated resist of immersion lithography such as material for a protective layer of laminated resist of immersion lithography, material for a resist layer of laminated resist of immersion lithography and material for anti-reflection film, material for other anti-reflection film, material for light emitting element, material for lens, material for optical device, material for display device, material for optical recording, material for transmitting optical signal (optical transfer medium), and materials for sealing members thereof. In addition, the polymer of the present invention can be used suitably as materials for medical use such as coating materials for jointing portions and filters for various medical devices.

Examples of light emitting element are EL element, light emitting polymer diode, light emitting diode, optical fiber laser, laser element, optical fiber, liquid crystal back light, photo-detector and the like, and these are applied to large-size display, illumination, liquid crystal, optical disc system, laser printer, laser for medical use, laser processing, printing, copying machine, etc. The polymer of the present invention is excellent in transparency, mold-processability and light fastness and is suitable for these applications.

Examples of applications of material for lens are condensing lens, pick-up lens, lens for glasses, lens for camera, Fresnel lens of projector, contact lens and the like. The polymer of the present invention is excellent in transparency, heat resistance and mold-processability and is suitable for these applications.

Examples of applications of optical material for optical device are optical wave-guide devices such as optical amplifying device, optical switch, optical filter, optical branching device and wavelength conversion device. Also, an optical circuit made by combining an optical branching device including N-branch wave-guide (N is an integer of 2 or more) with the above-mentioned devices is very useful in the future high-grade information communication society. The combination of these devices can be used for optical router, ONU, OADM, media converter, etc. With respect to optical wave-guide devices, any of flat type, strip type, ridge type and embedded type can be optionally selected. The polymer of the present invention is suitable for these applications since it is high in transparency within a wide wavelength range, is excellent in mold-processability and is low in a refractive index.

Examples of applications of optical material for display device are anti-reflection material, covering material of illumination, display protecting panel, transparent case, display panel, parts of automobiles, and the like. The polymer of the present invention is suitable for these applications since it is high in transparency within a wide wavelength range, is excellent in mold-processability and is low in a refractive index.

Examples of applications of optical recording material are a substrate for optical disc, matrix of volume holographic recording material, and the like. The polymer of the present invention is suitable for these applications since it is high in transparency within a wide wavelength range, is excellent in mold-processability and is low in a refractive index.

Examples of applications of material for transmitting optical signal (optical transfer medium) are heat-resistant optical transfer medium, core and/or clad material of plastic optical fiber formed from a core and clad, and the like. The polymer of the present invention is suitable for these applications since it has high glass transition temperature.

Though the polymer of the present invention is insoluble in water and exhibits high static and dynamic contact angle, it has such surface wettability as exhibiting solubility in alkaline solution. Therefore, there is a way of using the polymer for applications where bio-compatibility is required while inhibiting adsorption of bio-substances such as protein, and the polymer can be used as material for medical use such as coating materials for jointing portions and filters for various medical devices.

The following explanation is focused on the method of forming a resist pattern in the case of using the polymer as a material for the protective layer of the laminated resist for immersion lithography and a material for the resist layer of the laminated resist for immersion lithography.

The method of forming a resist pattern by using the polymer of the present invention as a material for the protective layer of the laminated resist is a method of forming a resist pattern by immersion lithography comprising:

(I) a step for forming a laminated resist for immersion lithography comprising a substrate and a photo-resist layer to be formed on the substrate,
(II) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(III) a step for treating the exposed laminated resist with a developing solution, and
the photo-resist layer is characterized by comprising the polymer of the present invention.

The laminated resist (hereinafter also referred to as “first laminated resist”) having the protective layer comprising the polymer of the present invention is used especially effectively in the exposing step of immersion lithography in which exposing is carried out with ultraviolet light having a wavelength of not less than 193 nm and pure water is used as a liquid medium.

Namely, in the first laminated resist of the present invention, the protective layer (L2) is further formed on the outermost surface of the resist film having the photo-resist layer (L1) containing a conventional resist material such as ArF or KrF resist, and by using the polymer of the present invention on the protective layer (L2), solubility in a developing solution can be remarkably improved, and the first laminated resist has good water repellency, light transmittance and water resistance.

In the first laminated resist, the protective layer (L2) forming the outermost layer need be transparent to light having a wavelength of not less than 193 nm.

By using such a protective layer, an immersion exposing process using pure water can be utilized, for example, for ArF lithography using light having a wavelength of 193 nm and also KrF lithography using light having a wavelength of 248 nm.

Concretely in the case of a wavelength of not less than 193 nm, an absorption coefficient is not more than 1.0 μm⁻¹, preferably not more than 0.8 μm⁻¹, more preferably not more than 0.5 μm⁻¹, most preferably not more than 0.3 μm⁻¹.

A too large absorption coefficient of the protective layer (L2) is not preferred since transparency of the whole laminated resist is lowered, thereby lowering resolution at forming a fine pattern and resulting in deterioration of a pattern form.

Also it is preferable that the protective layer (L2) is difficult to dissolve in pure water or is low in a dissolution rate in pure water while having good solubility in a developing solution, for example, a 2.38% aqueous solution of tetramethylammonium hydroxide (2.38% aqueous solution of TMAH).

Concretely with respect to the dissolution rate in a developing solution, the dissolution rate of the layer in a 2.38% aqueous solution of TMAH which is measured by QCM method explained infra is not less than 1 nm/sec, preferably not less than 10 nm/sec, more preferably not less than 100 nm/sec.

A too low dissolution rate in a developing solution is not preferred since resolution is lowered at forming a fine pattern and the pattern easily becomes in the form of T-top, thereby making it difficult to obtain an intended pattern form.

On the other hand, it is preferable that the protective layer (L2) is difficult to dissolve in pure water. According to the measurement by the QCM method, the dissolution rate of the layer in pure water is not more than 10 nm/min, preferably not more than 8 nm/min, more preferably not more than 5 nm/min, especially preferably not more than 2 nm/min.

A too high dissolution rate in pure water is not preferred since the protecting effect by the protective layer (L2) becomes insufficient and the effect of improvement in solving the above-mentioned problems becomes insufficient.

For the measurement of the dissolution rate in pure water, ion-exchanged water obtained by using a usual ion exchange membrane is used as pure water.

Also it is preferable that the protective layer (L2) has high water repellency to such an extent not to lower the dissolution rate in a developing solution remarkably.

For example, a water contact angle of the protective layer is preferably not less than 70°, more preferably not less than 75°, especially preferably not less than 80°. An upper limit thereof is preferably not more than 100°, more preferably not more than 95°, especially preferably not more than 90°.

If the water contact angle on the surface of the protective layer (L2) is too small, after coming into contact with pure water, water permeation becomes fast and water easily reaches the photo-resist layer (L1), resulting in insufficient protecting effect by the protective layer (L2). Therefore a too small water contact angle is not preferred.

On the contrary, a too large water contact angle on the surface of the protective layer (L2) is not preferred because the dissolution rate in a developing solution is remarkably decreased.

Further, the protective layer (L2) having a low water absorbing property (water absorbing rate) is preferred.

If the water absorbing property (water absorbing rate) is too high, after coming into contact with pure water, water permeation becomes fast and water easily reaches the photo-resist layer (L1), resulting in insufficient protecting effect by the protective layer (L2). Therefore a too high water absorbing property is not preferred.

For example, the water absorbing property (water absorbing rate) can be measured by the QCM method, and calculated as a weight increasing rate by water absorption (water absorbing rate).

The polymer of the present invention is used on the protective layer (L2) having these properties mentioned above.

In the first laminated resist of the present invention, the protective layer (L2) is formed by coating the coating composition comprising the polymer of the present invention on the photo-resist layer (L1) previously formed.

The coating composition forming the protective layer (L2) comprises the polymer of the present invention and a solvent.

It is preferable that the solvent is selected from those dissolving the polymer of the present invention uniformly, and a solvent having good film forming property is optionally selected and used.

Preferred examples of the solvent are cellosolve solvent, ester solvent, propylene glycol solvent, ketone solvent, aromatic hydrocarbon solvent, alcohol solvent, water and solvent mixture thereof. Further, fluorine-containing solvents such as fluorine-containing hydrocarbon solvents such as CH₃CCl₂F (HCFC-141b) and fluorine-containing alcohols may be used together for enhancing solubility of the polymer of the present invention and film forming property.

It is preferable that the solvent is selected from solvents which do not re-dissolve the lower photo-resist film (L1) previously formed. From this point of view, water and/or alcohols are preferred.

An amount of the solvent is selected depending on kind of solids to be dissolved, kind of a substrate to be coated, a target coating thickness and the like. From the viewpoint of easy coating, it is preferable that the solvent is used in such an amount that the concentration of the whole solids of the photo-resist composition is from 0.5 to 70% by weight, preferably from 1 to 50% by weight.

Among the solvents, water is not limited particularly. Preferred are distilled water, ion exchange water, water subjected to filtration and water subjected to various adsorption treatments to remove organic impurities and metal ion.

Alcohols are optionally selected from those which do not re-dissolve the lower photo-resist layer (L1), depending on kind of the photo-resist layer (L1). Generally lower alcohols are preferred, and concretely methanol, ethanol, isopropanol, n-propanol and the like are preferred.

In addition to these solvents, a water soluble organic solvent may be used together for the purpose of improving coatability, etc. to such an extent not to re-dissolve the photo-resist layer (L1).

A water soluble organic solvent is not limited particularly as far as it dissolves in an amount of not less than 1% by mass based on water. Preferred examples thereof are, for instance, ketones such as acetone and methyl ethyl ketone; esters of acetic acids such as methyl acetate and ethyl acetate; polar solvents such as dimethylformamide, dimethyl sulfoxide, methyl cellosolve, cellosolve acetate, butyl cellosolve, butyl carbitol and carbitol acetate; and the like.

An adding amount of the water soluble organic solvent to be added in addition to water or alcohol is from 0.1 to 50% by mass, preferably from 0.5 to 30% by mass, more preferably from 1 to 20% by mass, especially preferably from 1 to 10% by mass based on the total amount of the solvent.

To the coating composition forming the protective layer (L2) of the present invention may be added, as case demands, at least one selected from basic substances, for example, ammonia and organic amines. In this case, there is a case where an acidic OH group having a pKa value of not more than 11 becomes a hydrophilic derivative moiety, for example, in the form of ammonium salt, amine salt or the like in the coating composition.

With respect to the organic amines, preferred are water soluble organic amine compounds. Preferred examples thereof are, for instance, primary amines such as methylamine, ethylamine and propylamine; secondary amines such as dimethylamine and diethylamine; tertiary amines such as trimethylamine, triethylamine and pyridine; hydroxylamines such as monoethanolamine, propanolamine, diethanolamine, triethanolamine and tris(hydroxymethyl)aminomethane; quaternary ammonium compounds such as tetramethylammonium hydroxide, tetraethylammonium hydroxide, tetrapropylammonium hydroxide and tetrabutylammonium hydroxide; and the like.

Among them, from the viewpoint of increasing a rate of dissolution in a developing solution, preferred are hydroxylamines such as monoethanolamine, propanolamine, diethanolamine, triethanolamine and tris(hydroxymethyl)aminomethane, and particularly preferred is monoethanolamine.

Also, to the coating composition of the present invention for forming the protective layer (L2) may be added, as case demands, a defoaming agent, light absorbing agent, storage stabilizer, antiseptic agent, adhesion promoter, photoacid generator and the like.

In the coating composition of the present invention for forming the protective layer (L2), the content of the polymer of the present invention varies depending on kind and molecular weight of the polymer, kind and amount of additives, kind of a solvent and the like, and is optionally selected so that a proper viscosity enabling a thin coating film to be formed is obtained. For example, the content of the polymer is from 0.1 to 50% by mass, preferably from 0.5 to 30% by mass, more preferably from 1 to 20% by mass, especially preferably from 2 to 10% by mass based on the whole coating composition.

The coating composition is applied on the photo-resist layer (L1) to form the protective layer (L2) as an outermost layer of the laminated resist.

For the application, conventional methods are adopted. Examples of the suitable methods are rotary coating method, cast coating method, roll coating method and the like, and particularly a rotary coating method (spin coating method) is preferred.

A thickness of the protective layer varies depending on immersion exposing conditions, contact time with water and the like, and is optionally selected. The thickness is usually from 1 to 500 nm, preferably from 10 to 300 nm, more preferably from 20 to 200 nm, especially preferably from 30 to 100 nm.

Since transparency of the polymer of the present invention is high, a fine pattern can be formed even if the thickness of the protective layer is thick.

In the first laminated resist, the photo-resist layer (L1) is a layer formed using a conventional photo-resist composition on a substrate such as a wafer mentioned infra.

The photo-resist layer is a layer obtained by forming a film of, for example, a positive photo-resist containing, as main components, a novolak resin and diazonaphthoquinone (g-line or i-line lithography), a chemically amplifying positive or negative resist prepared using polyhydroxystyrene as a binder resin (KrF lithography), or a chemically amplifying positive photo-resist prepared using an acrylic polymer having an alicyclic structure in its side chain or an alicyclic polymer having a polynorbornene structure (ArF lithography).

A thickness of the photo-resist layer (L1) varies depending on kind and purpose of a device to be produced, conditions for etching and the like for production thereof and kind of the resist layer (degrees of transparency and dry etch resistance), and is optionally selected. The thickness is usually from 10 to 5,000 nm, preferably from 50 to 1,000 nm, more preferably from 100 to 500 nm.

The protective layer (L2) of the present invention is excellent in at least one of water repellency, water resistance and water-proof property at immersion exposing using pure water, as compared with conventional resists having a photo-resist layer as an outermost layer or an antireflection layer as an outermost layer. Therefore, the protective layer can be preferably applied especially to an immersion photolithography process using a chemically amplifying positive photo-resist (ArF lithography) prepared from an acrylic polymer having an alicyclic structure in its side chain or an alicyclic polymer having a polynorbornene structure, and purposes of obtaining precise pattern form, high dimensional accuracy of a pattern and reproducibility thereof are accomplished effectively.

Examples of the substrate in the first laminated resist are, for instance, a silicon wafer; a glass substrate; a silicon wafer or glass substrate provided with an organic or inorganic antireflection film; a silicon wafer which has steps and is provided with various insulating films, electrode and wiring on a surface thereof; a mask blank; a semiconductor wafer of III-V group compound such as GaAs or AlGaAs and a semiconductor wafer of II-VI group compound; a piezoelectric wafer of crystal, quartz or lithium tantalate and the like.

The formation of the resist is not limited to the case of forming the resist on a so-called substrate. The resist may also be formed on a specific layer such as an electrically conductive film, insulating film or the like which is formed on the substrate. Also, it is possible to form an antireflection film (lower antireflection layer), for example, DUV-30, DUV-32, DUV-42 and DUV-44 available from Brewer Science Co., Ltd. on the substrate. The substrate may be treated with an adhesion improver.

Next, an example of the method of producing the first laminated resist, namely the method of forming the laminated resist by providing the protective layer (L2) on the photo-resist layer (L1) and further the method of forming a fine pattern by immersion exposing using the laminated photo-resist is explained below by means of the drawing.

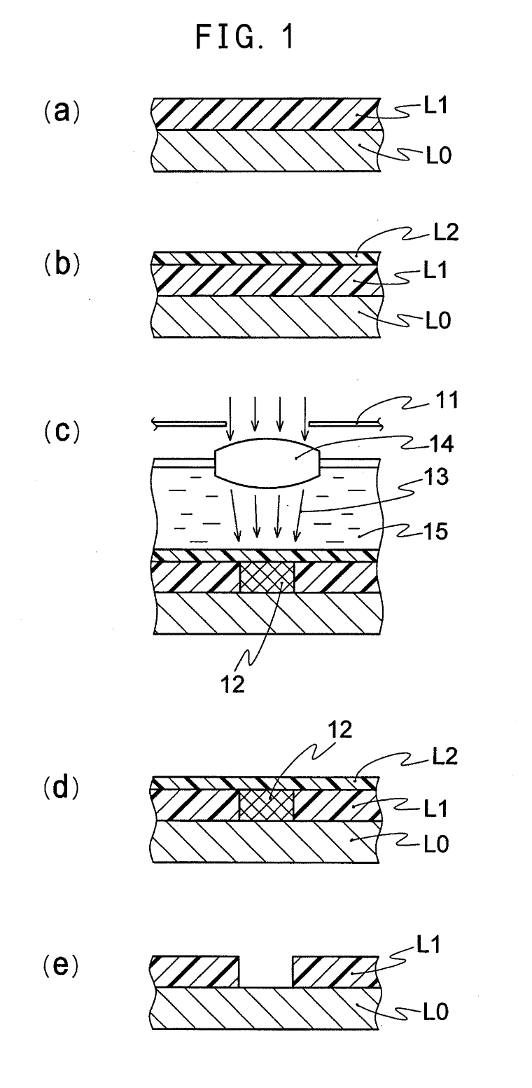

FIG. 1 is a diagrammatic view for explaining each step (a) to (e) of the method of forming the first laminated resist of the present invention and the method of forming a fine pattern by immersion exposing.

### (a) A Step for Forming the Photo-Resist Layer (L1):

First, as shown in FIG. 1(a), the photo-resist composition is coated on a substrate (L0) by a rotary coating method or the like in a coating thickness of from 10 to 5,000 nm, preferably from 50 to 1,000 nm, more preferably from 100 to 500 nm.

Next, pre-baking treatment is carried out at a pre-determined temperature of not more than 150° C., preferably from 80° to 130° C. to form the photo-resist layer (L1).

### (b) A Step for Forming the Protective Layer (L2):

As shown in FIG. 1(b), on the dried photo-resist layer (L1) is applied the coating composition containing the polymer of the present invention by a rotary coating method or the like. Then, pre-baking is carried out, as case demands, to form the protective layer (L2).

The pre-baking conditions are optionally selected for the purpose of evaporating the residual solvent in the protective layer (L2) and further forming a uniform thin film. For example, the pre-baking temperature is selected within a range from room temperature to 150° C., preferably from 40° to 120° C., more preferably from 60° to 100° C.

### (c) A Step for Immersion Exposing:

Subsequently, as shown in FIG. 1(c), a pattern is drawn on the laminated resist (L1+L2) by irradiating the resist with energy rays as shown by an arrow 13 through a mask 11 having a desired pattern and a reduction projection lens 14, thus selectively exposing a specific area 12.

In the present invention, the exposing is carried out in a state of pure water 15 being filled between the reduction projection lens 14 and the laminated resist.

In the first laminated resist, intended purposes of obtaining a precise pattern form, high dimensional accuracy of a pattern and reproducibility thereof are accomplished by an effect of the protective layer (L2) in a state of pure water being filled between the lens and the resist.

In this case, for example, g-line (436 nm wavelength), i-line (365 nm wavelength), KrF excimer laser (248 nm wavelength), ArF excimer laser (193 nm wavelength) and the like can be used as the energy rays (or chemical radiation), and resolution can be enhanced in the respective processes.

Particularly in the case of ArF excimer laser (193 nm wavelength), a high resolution effect by immersion exposing is exhibited more.

Subsequently, by carrying out post-exposure baking (PEB step) at a temperature of from 70° to 160° C., preferably from 90° to 140° C. for about 30 seconds to about 10 minutes, a latent image is formed on the exposed area 12 of the photo-resist layer (L1) as shown in FIG. 1(d). At this time, an acid generated by the exposing acts as a catalyst to decompose the dissolution-inhibiting group (protective group) in the photo-resist layer (L1), thereby increasing solubility in a developing solution and making the exposed area of the resist film soluble in a developing solution.

### (d) Developing Step:

Then, when the photo-resist layer (L1) after the post-exposure baking is subjected to developing with a developing solution, the un-exposed area of the photo-resist layer (L1) remains on the substrate because its solubility in the developing solution is low but the exposed area 12 is dissolved in the developing solution as mentioned above.

On the other hand, the upper protective layer (L2) is excellent in solubility in the developing solution irrespective of the exposed area and un-exposed area, and therefore is removed together with the exposed portion in the developing step.

A 2.38% by weight aqueous solution of tetramethylammonium hydroxide is preferably used as the developing solution. Also to the 2.38% by weight aqueous solution of tetramethylammonium hydroxide may be added a surfactant or alcohol such as methanol, ethanol, propanol or butanol in order to adjust wettability to the surfaces of the protective layer (L2) and photo-resist layer (L1).

Next, after flowing away the developing solution with pure water, lower alcohol or a mixture thereof, the substrate is dried and thus a desired resist pattern can be formed as shown in FIG. 1(e).

Also, when an intended fine pattern of an electrically conductive film or an insulating film is formed by using the so-formed fine resist pattern as a mask and etching a specific layer under the mask and then other steps are carried out, semiconductor devices and electronic devices can be produced. Since those steps are well known, explanation thereof is omitted.

The method of forming a resist pattern by using the polymer of the present invention as a material for the laminated resist is a method of forming a resist pattern by immersion lithography comprising:

(Ia) a step for forming a laminated resist for immersion lithography comprising a substrate, a photo-resist layer to be formed on the substrate and a protective layer to be formed on the photo-resist layer,
(IIa) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(IIIa) a step for treating the exposed laminated resist with a developing solution, and
the photo-resist layer and/or protective layer comprises the polymer of the present invention.

The laminated resist of this method is a laminated resist for immersion lithography using ultraviolet light having a wavelength of not less than 193 nm for exposing and comprises a substrate and a photo-resist layer (L3) provided on the substrate. This laminated resist is characterized in that the photo-resist layer (L3) is formed on a substrate as an outermost surface of the laminated resist and contains the photoacid generator and the polymer having the protective group Y² which can change to an alkali soluble group by dissociation with an acid (hereinafter also referred to as “second laminated resist”).

The inventors of the present invention have found that when the second laminated resist having the photo-resist layer (L3) as an outermost surface is used for immersion photolithography process using pure water as a liquid medium, it is possible to make an improvement in solving a problem with a pattern failure and defect in an immersion exposing process which has been difficult to solve in the case of a film surface of conventional ArF resist or KrF resist.

In the present invention, the photo-resist layer (L3) containing the acid-dissociative polymer is excellent in at least one of water repellency, water resistance and water-proof property, and therefore it can be considered that even if the photo-resist layer (L3) is used as an outermost surface and comes into contact with pure water, diffusion and elution of the photo-acid generator contained in the photo-resist layer (L3) and diffusion and elution of a quencher can be inhibited.

In the second laminated resist, the photo-resist layer (L3) containing the above-mentioned acid-dissociative polymer may be applied directly to the substrate or may be applied to a photo-resist layer (L3-1) of a conventional ArF resist or KrF resist as a layer having a protective function in the same manner as mentioned above.

Especially it is preferable that water repellency of the photo-resist layer (L3) forming an outermost layer is higher to such an extent not to lower developing characteristics significantly after the exposing.

For example, a water contact angle of the photo-resist layer (L3) is preferably not less than 70°, more preferably not less than 75°, especially preferably not less than 80°. An upper limit thereof is preferably not more than 110°, more preferably not more than 100°, especially preferably not more than 90°.

If the water contact angle of the photo-resist layer (L3) surface is too small, after coming into contact with pure water, water permeation becomes fast, thereby increasing water absorption and swelling of the photo-resist layer (L3) or causing elution of additives such as a photoacid generator and amines contained in the photo-resist layer (L3), which has an adverse effect on resolution and form of a fine pattern. Therefore a too small water contact angle is not preferred. Also a too small water contact angle is not preferred because when the photo-resist layer (L3) of the present invention forming an outermost layer is formed on the conventional photo-resist layer (L3-1), water easily reaches the lower photo-resist layer (L3-1), which has an adverse effect on resolution and form of a fine pattern like the case mentioned above.

Also, a too large water contact angle on the photo-resist layer (L3) surface is not preferred because at developing after the exposing, the rate of dissolution in a developing solution of the exposed portion is decreased, which has an adverse effect on resolution and form of a fine pattern.

Further, the photo-resist layer (L3) of the outermost surface is preferably one having a low water absorbing property (water absorbing rate).

If the water absorbing property (water absorbing rate) is too high, after coming into contact with pure water, water permeation becomes fast and a rate of water permeation into the photo-resist layer (L3) is increased. Therefore, a too high water absorbing property is not preferred.

If the water absorbing property (water absorbing rate) of the photo-resist layer (L3) is too high, after coming into contact with pure water, elution of additives such as a photoacid generator and amines contained in the photo-resist layer (L3) occurs, which has an adverse effect on resolution and form of a fine pattern. Therefore, a too high water absorbing rate is not preferred. Also a too high water absorbing rate is not preferred because when the photo-resist layer (L3) of the present invention forming an outermost layer is formed on the conventional photo-resist layer (L3-1), water easily reaches the lower photo-resist layer (L3-1), which has an adverse effect on resolution and form of a fine pattern like the case mentioned above.

For example, the water absorbing property (water absorbing rate) can be measured by the QCM method, and calculated as a weight increasing rate by water absorption (water absorbing rate).

In the second laminated resist, it is necessary that the photo-resist layer (L3) forming an outermost layer is transparent to light having a wavelength of not less than 193 nm.

Accordingly an immersion exposing process using pure water can be utilized usefully, for example, even in ArF lithography using 193 nm wavelength and KrF lithography using 248 nm wavelength.

Concretely in the case of a wavelength of not less than 193 nm, an absorption coefficient is not more than 1.0 μm⁻¹, preferably not more than 0.8 μm⁻¹, more preferably not more than 0.5 μm⁻¹, most preferably not more than 0.3 μm⁻¹.

A too large absorption coefficient of the photo-resist layer (L3) is not preferred because transparency of the whole laminated resist is lowered, resulting in lowering of resolution at forming a fine pattern and deterioration of a pattern form.

It is important that the acid-dissociative polymer contained in the photo-resist layer (L3) of the second laminated resist has the protective group Y² which can be changed to an alkali soluble group by dissociation with an acid. Namely, the polymer is one being capable of acting as a positive resist. Accordingly, the photo-resist layer (L3) further contains the photoacid generator as essential component and contains, as case demands, amines and other additives necessary for a resist.

The protective group Y² contained in the acid-dissociative polymer is a functional group (—OR) which can make the polymer soluble in alkali by an action of an acid though the polymer is insoluble or less soluble in alkali before reaction with an acid. This change in solubility in alkali makes the polymer usable as a base polymer for a positive resist.

Examples of the protective group which can be used preferably are:

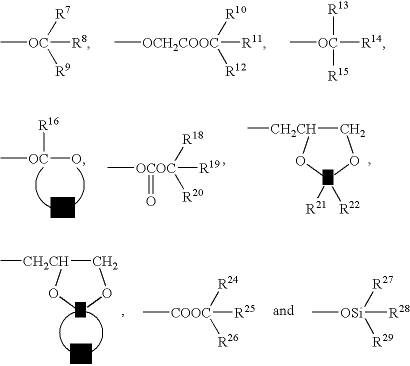

wherein R⁷, R⁸, R⁹, R¹⁰, R¹¹, R¹², R¹⁴, R¹⁸, R¹⁹, R²⁰, R²¹, R²², R²⁴, R²⁵, R²⁶, R²⁷, R²⁸ and R²⁹ are the same or different and each is a hydrocarbon group having 1 to 10 carbon atoms; R¹³, R¹⁵ and R¹⁶ are the same or different and each is H or a hydrocarbon group having 1 to 10 carbon atoms; and R¹⁷ and R²³ are the same or different and each is a divalent hydrocarbon group having 2 to 10 carbon atoms. More concretely there are preferably:

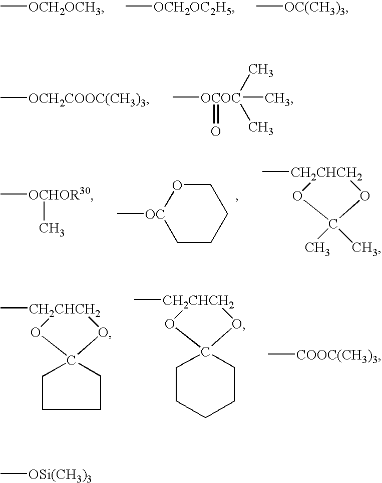

and the like, wherein R³⁰ is an alkyl group having 1 to 10 carbon atoms.

Among the above-mentioned protective groups Y², preferred is at least one of protective groups Y³ which can be converted to OH group by an acid.

Preferred examples of the protective groups Y³ which can be converted to OH group by an acid are groups represented by:

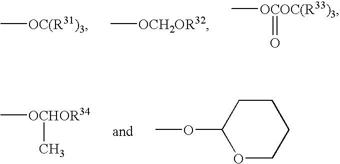

wherein R³¹, R³², R³³ and R³⁴ are the same or different and each is an alkyl group having 1 to 5 carbon atoms.

More concretely there are preferably:

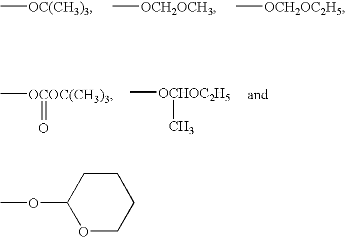

and among these, preferred are:

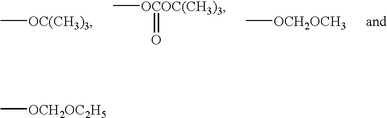

because of good reactivity with an acid, and —OC(CH₃)₃, —OCH₂OCH₃ and —OCH₂OC₂H₅ are preferred because of good transparency.

Among the protective groups Y³ which can be converted to OH group by an acid, preferred are those which can be converted to OH showing acidity of not more than 11 in a pKa value by an acid, further preferably OH group having a pKa value of not more than 10, particularly preferably OH group having a pKa value of not more than 9.

Such protective groups are preferred because developing characteristics after the exposing become good and a fine pattern of high resolution can be obtained.

It is preferable that a fluorine-containing alkyl group or a fluorine-containing alkylene group is bonded to the carbon atom bonded directly to the protective groups Y³ which can be converted to OH group, and preferred is a moiety represented by the following formula:

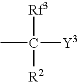

wherein Rf³ is a fluorine-containing alkyl group which has 1 to 10 carbon atoms and may have ether bond; R² is selected from hydrogen atom, hydrocarbon groups having 1 to 10 carbon atoms and fluorine-containing alkyl groups which have 1 to 10 carbon atoms and may have ether bond.

It is preferable that R² is a fluorine-containing alkyl group which has 1 to 10 carbon atoms and may have ether bond.

It is further preferable that both of Rf³ and R² are perfluoroalkyl groups, and concretely preferred are moieties of:

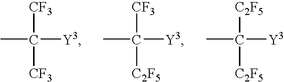

and the like.

Further, from the viewpoint of water solubility and solubility in a developing solution, more preferred is a moiety represented by the following formula:

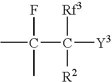

wherein Rf³ is a fluorine-containing alkyl group which has 1 to 10 carbon atoms and may have ether bond; R² is selected from hydrogen atom, hydrocarbon groups having 1 to 10 carbon atoms and fluorine-containing alkyl groups which have 1 to 10 carbon atoms and may have ether bond. Concretely preferred are moieties of:

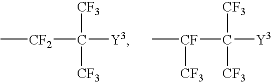

and the like.

It is preferable that the fluorine content of the fluorine-containing acid-dissociative polymer having the protective group Y² is not less than 30% by mass, more preferably not less than 40% by mass, especially preferably not less than 50% by mass.

A too low fluorine content is not preferred because water repellency is lowered and water absorption is increased too much.

On the other hand, an upper limit of the fluorine content is 75% by mass, preferably 70% by mass, more preferably 65% by mass.

A too high fluorine content is not preferred because water repellency of the coating film becomes too high, thereby decreasing a rate of dissolution in a developing solution and deteriorating reproducibility of the rate of dissolution in a developing solution.

The acid-dissociative polymer having the protective group Y² and used for the photo-resist layer (L3) formed as the outermost surface of the second laminated resist is obtained by replacing —OR′ of the above-mentioned polymer of the present invention by at least one of the above-mentioned protective groups Y², and as a result, can work as a positive resist. For introducing the protective group Y² to the polymer of the present invention, usual method can be adopted.

In the second laminated resist, the photo-resist layer (L3) comprises the photoacid generator in addition to the above-mentioned acid-dissociative polymer.

Preferred examples of the photoacid generator are the same as the examples of the photoacid generator (b) described in International Publication No. WO 01/74916. Those photoacid generators can also be used effectively in the present invention.

The photoacid generator is a compound which generates an acid or a cation by irradiation of light. Examples thereof are, for instance, organic halogen compounds, sulfonic acid esters, onium salts (particularly fluoroalkyl onium salts having iodine, sulfur, selenium, tellurium, nitrogen or phosphorus as a center element), diazonium salts, disulfone compounds, sulfonediazides and mixtures thereof.

More preferred examples thereof are as follows.

### (1) TPS Compound:

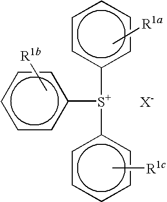

wherein X⁻ is PF₆⁻, SbF₆⁻, CF₃SO₃⁻, C₄F₉SO₃⁻ or the like; R^{1a}, R^{1b} and R^{1c} are the same or different and each is CH₃O, H, t-Bu, CH₃, OH or the like.

### (2) DPI Compound:

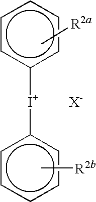

wherein X⁻ is CF₃SO₃⁻, C₄F₉SO₃⁻, CH₃-ph-SO₃⁻, SbF₆⁻,

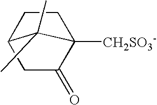

or the like; R^{2a} and R^{2b} are the same or different and each is H, OH, CH₃, CH₃O, t-Bu or the like.

### (3) Sulfonate Compound:

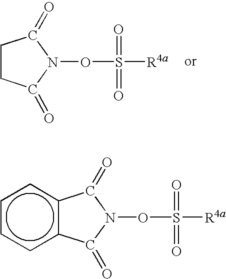

wherein R^{4a} is:

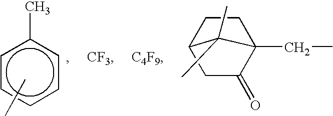

or the like.

Usually the photo-resist layer (L3) is formed, for example, by applying the resist composition prepared by dissolving the acid-dissociative polymer and the above-mentioned photoacid generator in the solvent and coating the composition.

In the second laminated resist, the content of photoacid generator used for the resist composition for forming the photo-resist layer (L3) is preferably from 0.1 to 30 parts by weight, more preferably from 0.2 to 20 parts by weight, most preferably from 0.5 to 10 parts by weight based on 100 parts by weight of the acid-dissociative polymer.

If the content of photoacid generator is lower than 0.1 part by weight, sensitivity is lowered, and if the content of photoacid generator is more than 30 parts by weight, an amount of light absorbed by the photoacid generator is increased and light does not reach a substrate sufficiently and therefore resolution is easily lowered.

Also to the resist composition for forming the photo-resist layer (L3) may be added an organic base being capable of acting as a base on an acid generated from the photoacid generator. Examples of preferred organic base are the same as those exemplified in International Publication No. WO 01/74916. Those organic bases can also be used effectively in the present invention.

The organic base is concretely an organic amine compound selected from nitrogen-containing compounds. Examples thereof are, for instance, pyridine compounds, pyrimidine compounds, amines substituted by a hydroxyalkyl group having 1 to 4 carbon atoms, amino phenols and the like. Particularly preferred are hydroxyl-containing amines.

Examples thereof are butylamine, dibutylamine, tributylamine, triethylamine, tripropylamine, triamylamine, pyridine and the like.

The content of organic base in the resist composition for forming the photo-resist layer (L3) is preferably from 0.1 to 100% by mole, more preferably from 1 to 50% by mole based on the content of photoacid generator. If the content of organic base is lower than 0.1% by mole, resolution is lowered, and if the content of organic base is more than 100% by mole, sensitivity tends to be lowered.

The resist composition may contain, as case demands, additives disclosed in International Publication No. WO 01/74916, for example, various additives which have been usually used in this field, such as dissolution inhibitor, sensitizer, dye, adhesion betterment material and water storage material.

Also in the resist composition for forming the photo-resist layer (L3) of the second laminated resist, examples of the preferred solvent are the same as those of the solvent exemplified in International Publication No. WO 01/74916. Those solvents can also be used effectively in the present invention.

Preferred examples thereof are cello solve solvents, ester solvents, propylene glycol solvents, ketone solvents, aromatic hydrocarbon solvents and solvent mixtures thereof. Also in order to enhance solubility of the acid-dissociative polymer, fluorine-containing solvents such as fluorine-containing hydrocarbon solvents such as CH₃CCl₂F (HCFC-141b) and fluorine-containing alcohols may be used together.

The amount of solvent is selected depending on kind of solids to be dissolved, kind of a substrate to be coated, an intended coating thickness, etc. From the viewpoint of easy coating, it is preferable that the solvent is used in such an amount that the concentration of the whole solids of the photo-resist composition is from 0.5 to 70% by weight, preferably from 1 to 50% by weight.

In the second laminated resist, the first of the preferred laminated resist is a laminated resist (X1) having a layer construction comprising a substrate and the photo-resist layer (L3) which contains the acid-dissociative polymer and is formed on the substrate.

In the laminated resist (X1), substantially only the photo-resist layer (L3) is laminated on the substrate. The photo-resist layer (L3) itself has high transparency to ultraviolet light having a wavelength of not less than 193 nm, and acts as a positive resist in a lithography process using such ultraviolet light and makes it possible to form a good pattern. Further the photo-resist layer (L3) is preferred since an adverse effect due to water used in immersion lithography can be reduced to a minimum.

In the laminated resist (X1), a thickness of the photo-resist layer (L3) varies depending on kind and purpose of a device to be produced, conditions of processing, e.g. etching for production thereof and kind of the resist layer (degrees of transparency and dry etch resistance), and is optionally selected. The thickness is usually from 10 to 5,000 nm, preferably from 50 to 1,000 nm, more preferably from 100 to 500 nm.

In the second laminated resist, the second of the preferred laminated resist is a laminated resist (X2) having a layer construction comprising a substrate, a photo-resist layer (L3-1) formed previously on the substrate and the photo-resist layer (L3) which contains the acid-dissociative polymer and is formed on the photo-resist layer (L3-1).

This laminated resist (X2) is produced by laminating the photo-resist layer (L3) which contains the acid-dissociative polymer and functions as a protective layer against water, on the photo-resist layer (L3-1) of a conventional resist material, and both of the photo-resist layers (L3-1) and (L3) are subjected to pattern formation at the same time by the exposing and developing steps.

The photo-resist layer (L3-1) in the laminated resist is a layer formed by using a conventional photo-resist composition, for example, a layer obtained by forming a film by using a positive photo-resist containing, as main components, a novolak resin and diazonaphthoquinone (g-line or i-line lithography), a chemically amplifying positive or negative resist prepared using polyhydroxystyrene as a binder resin (KrF lithography), a chemically amplifying positive photo-resist prepared using an acrylic polymer having an alicyclic structure in its side chain or an alicyclic polymer having a polynorbornene structure (ArF lithography) or the like.

In the case of using for immersion lithography of the present invention, preferred are a chemically amplifying positive resist prepared using polyhydroxystyrene as a binder resin and a chemically amplifying positive photo-resist prepared using an acrylic polymer having an alicyclic structure in its side chain or an alicyclic polymer having a polynorbornene structure, and particularly preferred is a chemically amplifying positive photo-resist prepared using an acrylic polymer having an alicyclic structure in its side chain or an alicyclic polymer having a polynorbornene structure.

In the laminated resist (X2), a thickness of the photo-resist layer (L3) varies depending on kind of the acid-dissociative polymer, immersion exposing conditions, contact time with water and the like, and is optionally selected. The thickness is usually from 1 to 500 nm, preferably from 10 to 300 nm, more preferably from 20 to 200 nm, especially from 30 to 100 nm.

In the laminated resist (X2), a thickness of the photo-resist layer (L3-1) varies depending on kind and purpose of a device to be produced, conditions of processing, e.g. etching for production thereof and kind of the resist layer (degrees of transparency and dry etch resistance), and is optionally selected. The thickness is usually from 10 to 5,000 nm, preferably from 50 to 1,000 nm, more preferably from 100 to 500 nm.

This laminated resist (X2) can solve problems attributable to water at immersion exposing while utilizing dry etch resistance and lithographic characteristics (for example, film forming property, sensitivity, resolution and pattern form) of the lower photo-resist layer (L3-1) though the problems could not be solved sufficiently only by the photo-resist layer (L3-1).

Also, the laminated resist (X2) is preferred since the outermost photo-resist layer (L3) itself containing the acid-dissociative polymer can be formed into a pattern having the same form as that of the photo-resist layer (L3-1), thereby enabling the form and roughness of the pattern surface after the developing to be enhanced.

Examples of the substrate in the second laminated resists (X1) and (X2) of the present invention are a silicon wafer; a glass substrate; a silicon wafer or glass substrate provided with an organic or inorganic antireflection film; a silicon wafer which has steps and is provided with various insulating films, electrode and wiring on the surface thereof; a mask blank; a semiconductor wafer of III-V group compound such as GaAs or AlGaAs and a semiconductor wafer of II-VI group compound; a piezoelectric wafer of crystal, quartz or lithium tantalate, and the like.

The formation of the resist is not limited to the case of forming the resist on a so-called substrate. The resist may also be formed on a specific layer such as an electrically conductive film, insulating film or the like which is formed on the substrate. Also it is possible to form an antireflection film (lower antireflection layer), for example, DUV-30, DUV-32, DUV-42 and DUV44 available from Brewer Science Co., Ltd. on the substrate. The substrate may be treated with an adhesion improver.

With respect to the method of forming the photo-resist layer (L3) on the substrate, the method of forming the laminated resist by providing the photo-resist layer (L3) on the photo-resist layer (L3-1) and further the method of forming a fine pattern by immersion exposing by using the laminated resists (X1) and (X2), there can be similarly adopted the mentioned method of forming the laminated resist by providing the protective layer (L2) on the photo-resist layer (L1) and further the mentioned method of forming a fine pattern by immersion exposing by using the obtained laminated photo-resist.

For example, with respect to the laminated resist (X1), a fine pattern can be formed by employing conventional method of forming a resist layer and carrying out steps including an immersion exposing step.

Also, the laminated resist (X2) can be formed by using the photo-resist layer (L3-1) instead of the photo-resist layer (L1) and using the photo-resist layer (L3) instead of the protective layer (L2) in the same manner as mentioned supra, and a fine pattern can be formed by carrying out steps including an immersion exposing step by using the obtained laminated resist in the same manner as mentioned supra.

### EXAMPLES

The present invention is then explained concretely by means of examples, but is not limited to them.

Equipment and measuring conditions used for evaluating physical properties are as follows.

### (1) NMR

NMR measuring equipment: available from BRUKER CO., LTD.
Measuring conditions of ¹H-NMR: 400 MHz (tetramethylsilane=0 ppm)
Measuring conditions of ¹⁹F-NMR: 376 MHz (trichlorofluoromethane=0 ppm)
(2) A number average (a weight average) molecular weight is calculated from the data measured by gel permeation chromatography (GPC) by using GPC HLC-8020 available from Toso Kabushiki Kaisha and columns available from Shodex (one GPC KF-801, one GPC KF-802 and two GPC KF-806M were connected in series) and flowing tetrahydrofuran (THF) as a solvent at a flowing rate of 1 ml/min.

### Example 1

### Synthesis of 5,5,5-trifluoro-4-hydroxy-4-(trifluoromethyl)pentan-2-yl 2-fluoroacrylate

A four-necked flask equipped with a nitrogen-feeding tube, dropping funnel, thermometer, silica gel drying tube, septum rubber cap and stirrer tip was dried, and into the flask were added 22.5 g (100 mmol) of 5,5,5-trifluoro-4-hydroxy-4-(trifluoromethyl)pentan-2-ol), 79 g (100 mmol) of pyridine and 50 ml of THF, followed by cooling on ice-water bath. Thereto was slowly added dropwise 10 g (110 mmol) of 2-fluoroacrylic acid fluoride at the inside temperature of not more than 5° C. with stirring. Then, stirring was continued overnight at room temperature under nitrogen atmosphere. Into the reaction mixture were added 100 ml of water and 50 ml of diisopropyl ether for separation of the solution. Washing was carried out with a saturated aqueous solution of sodium hydrogencarbonate two times, an aqueous solution of diluted hydrochloric acid once and saturated brine two times, followed by drying with anhydrous magnesium sulfate. The obtained solution was condensed and distilled under reduced pressure in the presence of hydroquinone to obtain a colorless transparent liquid. According to evaluation of a structure with ¹⁹F-NMR and ¹H-NMR, the obtained product was identified as 5,5,5-trifluoro-4-hydroxy-4-(trifluoromethyl) pentan-2-yl 2-fluoroacrylate. Yield was 19.5 g (65.7%), and a boiling point was 57° to 65° C. (1.0 mmHg).

¹⁹F-NMR (acetone-d6, ppm): −75.7 (3F), −76.9 (3F), −117.4 (1F)

¹H-NMR (acetone-d6, ppm): 1.41 (3H, CH₃), 2.29 (1H, CH₂), 2.54 (1H, CH₂), 5.44 (2H, H₂C═), 5.71 (1H, CH), 6.92 (1H, OH)

### Example 2

### Synthesis of homopolymer of 5,5,5-trifluoro-4-hydroxy-4-(trifluoromethyl)pentan-2-yl 2-fluoroacrylate

A three-necked flask equipped with a nitrogen-feeding tube, pressure reducing line, thermometer, septum rubber cap and stirrer tip was dried, and into the flask were added 3.0 g (10 mmol) of 5,5,5-trifluoro-4-hydroxy-4-(trifluoromethyl) pentan-2-yl 2-fluoroacrylate) synthesized in Example 1 and 15 ml of THF, followed by cooling on dry ice-acetone bath. After adding 60 mg (0.4 mmol) of azobis isobutyronitrile (AIBN) thereto, pressure was reduced with stirring to conduct deoxidation. Then, after replacing by nitrogen, the mixture was heated up to 60° C. on water bath, and stirring was continued for three hours. After the mixture was brought to room temperature and stirred for twenty hours, the reaction mixture was poured into 300 ml of n-hexane with stirring and a target resin was obtained by re-precipitation. According to evaluation of a structure with ¹⁹F-NMR and ¹H-NMR, the obtained product was identified as a homopolymer of 5,5,5-trifluoro-4-hydroxy-4-(trifluoromethyl)pentan-2-yl 2-fluoroacrylate. A number average molecular weight measured with GPC based on styrene was 27,580, and a weight average molecular weight was 30,880. Yield was 2.7 g (90%).

¹⁹F-NMR (acetone-d6, ppm): −75.6 (3F), −76.9 (3F), −161.2 to −167.8 (1F)

¹H-NMR (acetone-d6, ppm): 1.42 (3H, CH₃), 2.24 (1H, CH₂), 2.47 (3H, CH₂), 5.22 (1H, CH), 6.72 (1H, OH)

### Comparative Example 1

### Synthesis of homopolymer of 5,5,5-trifluoro-4-hydroxy-4-(trifluoromethyl)pentan-2-yl methacrylate

5,5,5-Trifluoro-4-hydroxy-4-(trifluoromethyl)pentan-2-yl methacrylate was prepared in the same manner as in Example 1 except that methacrylic acid fluoride was used instead of 2-fluoroacrylic acid fluoride. To 3.0 g (10 mmol) of this monomer was added 15 ml of THF, followed by cooling on dry ice-acetone bath. Then, after adding 60 mg (0.4 mmol) of AIBN thereto, pressure was reduced with stirring to carry out deoxidation. After replacing by nitrogen gas, the mixture was heated to 55° C. on water bath, and stirring was continued for two hours. Then, after the mixture was brought to room temperature and stirred for twenty hours, the reaction mixture was poured into 300 ml of n-hexane with stirring, and a target resin was obtained by re-precipitation. According to evaluation of a structure with ¹⁹F-NMR and ¹H-NMR, the obtained product was identified as a homopolymer of 5,5,5-trifluoro-4-hydroxy-4-(trifluoromethyl)pentan-2-yl methacrylate. A number average molecular weight measured with GPC based on styrene was 18,300, and a weight average molecular weight was 23,130. Yield was 0.84 g (28%).

### Comparative Example 2

A homopolymer of 5,5,5-trifluoro-4-hydroxy-4-(trifluoromethyl)pentan-2-yl methacrylate was prepared under the same reaction conditions as in Comparative Example 1 except that the solvent (THF) was not used. A number average molecular weight measured with GPC based on styrene was 207,120, and a weight average molecular weight was 295,720. Yield was 2.1 g (70%).

### Test Example 1

A static contact angle, advancing contact angle, receding contact angle and dropping angle were examined by using the polymers of Example 2, Comparative Example 1 and Comparative Example 2 by the following methods. The results are shown in Table 1.

### Preparation of Test Piece:

10% by mass methyl amyl ketone (MAK) solutions of the polymers prepared in Example 2, Comparative Example 1 and Comparative. Example 2, respectively are applied to a glass substrate by spin coating (300 rpm, 3 sec; 2,000 rpm, 25 sec) and then dried at 110° C. for 180 sec to prepare test pieces.

### Static Contact Angle:

A static contact angle is determined by dropping 2 μl each of water and n-hexadecane on the polymer film surface of the test piece placed flatly by using a micro syringe and then taking a static picture with a video microscope one second after the dropping.

### Advancing Contact Angle, Receding Contact Angle, Dropping Angle:

20 μl of water and 5 μl of n-hexadecane are dropped on the polymer film surface of the test piece placed flatly by using a micro syringe and the test piece is inclined at a rate of 2° per sec. A motion until a droplet begins to drop is recorded as a moving picture with a video microscope. The moving picture is played back and an angle when the droplet starts dropping is assumed to be a dropping angle. A contact angle of the front side of the droplet at a dropping angle is assumed to be a advancing contact angle, and a contact angle of the opposite side of the droplet is assumed to be a receding contact angle.

### Test Example 2

A rate of dissolution in a standard developing solution was examined by using the polymers of Example 2, Comparative Example 1 and Comparative Example 2 by the following methods. The results are shown in Table 1.

### Preparation of Test Piece:

10% by mass MAK solutions of the polymers obtained in Example 2, Comparative Example 1 and Comparative Example 2, respectively are applied to a 24 mm diameter quarts crystal oscillation panel coated with gold by spin coating (300 rpm, 5 sec; 2,000 rpm, 30 sec), and dried at 110° C. for 90 seconds to make an about 100 nm thick coating film of the polymer.

### Measurement of a Rate of Dissolution in a Standard Developing Solution:

A rate of dissolution in water is measured by quarts crystal oscillation method (QCM method) by using a 2.38% aqueous solution of tetramethylammonium hydroxide as a standard developing solution. A coating film thickness is calculated by converting the number of oscillations of the quarts crystal oscillation panel.

The quarts crystal oscillation panel of the test piece prepared above is dipped in pure water. A change in a coating film thickness with a lapse of time after the dipping is measured by a change in the number of oscillations and a rate of dissolution (nm/sec) per unit time is calculated (Reference bulletin: Advances in Resist Technology and Proceedings of SPIE Vol. 4690, 904 (2002)).

### Test Example 3

A glass transition temperature (Tg) and a thermal decomposition temperature (Td) were measured by using the polymers of Example 2, Comparative Example 1 and Comparative Example 2. The results are shown in Table 1.

### Glass Transition Temperature (Tg):

Elevation of temperature, lowering of temperature and elevation of temperature (the second elevation of temperature is called second run) are carried out at a temperature elevating or lowering rate of 10° C./min within a range from 30° C. to 150° C. by using a differential scanning calorimeter (RTG220 available from SEIKO), and an intermediate point of a heat absorption curve of the second run is assumed to be Tg (° C.).

### Thermal Decomposition Temperature (Td):

A temperature where the weight reduction of 5% by mass begins is measured at a heating rate of 10° C./min using a thermogravimeter TGA-50 available from Shimadzu Corporation.

### Test Example 4

Transmittance and refractive index were measured by using the polymers of Example 2, Comparative Example 1 and Comparative Example 2 by the following method. The results are shown in Table 1.

### Preparation of Test Piece:

10% by mass MAK solution of the polymers obtained in Example 2, Comparative Example 1 and Comparative Example 2 were coated on a 8 inch silicon wafer with a spin coater while rotating the wafer at 300 rpm for three seconds and then at 4,000 rpm for twenty seconds to form a film so that the coating thickness becomes about 100 nm after drying.

### Measurement of Refractive Index:

Transmittance (k value) and refractive index in each wavelength light and film thickness are measured with a spectroscopic ellipsometer (VASE ellipsometer available from J. A. Woollam).

TABLE 1

| | | Polymer | | |
|---|---|---|---|---|
| | | | Com. | Com. |
| | | Ex. 2 | Ex. 1 | Ex. 2 |
| | Static contact angle (degree) | | | |
| Test | Water | 84 | 86 | 86 |
| Ex. 1 | n-Hexane | 46 | 43 | 43 |
| | Advancing contact angle (degree) | | | |
| | Water | 93 | 94 | 93 |
| | Receding contact angle (degree) | | | |
| | Water | 69 | 71 | 70 |
| | Dropping angle (degree) | | | |
| | Water | 31 | 28 | 29 |
| Test | Dissolution rate in standard | 377 | 88 | 37 |
| Ex. 2 | developing solution (nm/sec) | | | |
| Test | Glass transition temperature | 94 | 88 | 95 |
| Ex. 3 | (° C.) | | | |
| | Thermal decomposition temperature | 273 | 248 | 282 |
| | (° C.) | | | |
| Test | k value (589 nm) | 0.000 | 0.002 | 0.000 |
| Ex. 4 | Refractive index (589 nm) | 1.366 | 1.404 | 1.404 |

### Example 3

### Formation of Laminated Resist

### (1) Formation of Photo-Resist Layer (L1)

A photo-resist TArF-P6071 for ArF lithography (available from Tokyo Ohka Kogyo Kabushiki Kaisha) was coated on a 8-inch silicon substrate with a spin coater while changing the number of revolutions to adjust the coating thickness to be 200 to 300 nm, followed by pre-baking at 130° C. for 60 seconds to form the photo-resist layer (L1).

### (2) Formation of Protective Layer (L2)

On the photo-resist layer (L1) formed in above (1) was coated the coating composition prepared in Example 2 and containing the homopolymer with a spin coater while rotating the wafer firstly at 300 rpm for three seconds and then at 4,000 rpm for twenty seconds and adjusting the coating thickness to be about 100 nm to form the protective layer (L2). Thus the laminated photo-resist was formed.

### (3) Developing

With respect to the laminated resists obtained in (2) above, stationary puddle-developing was carried out at 23° C. for 60 seconds by using a standard developing solution of 2.38% by mass tetramethylammonium hydroxide and then rinsing with pure water was carried out.

As a result, it was confirmed that the protective layer (L2) had been completely eliminated.

### INDUSTRIAL APPLICABILITY

The present invention can provide the fluorine-containing polymer having large static and dynamic water contact angles and greatly improved dissolution rate in a developing solution, the monomer providing the polymer, and the method of forming a resist pattern by immersion lithography using the fluorine-containing polymer.

Also, there can be provided the polymer applicable to various optical materials, for example, reflection preventing film, light-emitting element material, material for lens, materials for optical devices, materials for display, optical recording material, material for transmitting optical signal (optical transfer medium), and materials for sealing members thereof.

1. A polymerizable fluorine-containing monomer represented by the formula (1): 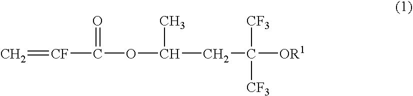
wherein R¹ is hydrogen atom or a monovalent saturated or unsaturated hydrocarbon group of 1 to 15 carbon atoms, said hydrocarbon group may be chain or cyclic structure and may have oxygen atom, nitrogen atom, sulfur atom or halogen atom.

2. A fluorine-containing polymer represented by the formula (I):
-(M)-(N)- (I)
wherein M is a structural unit derived from a polymerizable monomer represented by the formula (1): 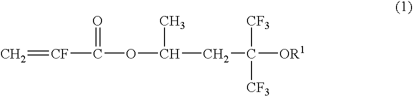
wherein R¹ is hydrogen atom or a monovalent saturated or unsaturated hydrocarbon group of 1 to 15 carbon atoms, said hydrocarbon group may be chain or cyclic structure and may have oxygen atom, nitrogen atom, sulfur atom or halogen atom; N is a structural unit derived from a monomer being copolymerizable with the monomer represented by the formula (1), and
the structural unit M is contained in an amount of 1 to 100% by mole and the structural unit N is contained in an amount of 0 to 99% by mole.

3. A method of forming a resist pattern by immersion lithography comprising:
(I) a step for forming a laminated resist for immersion lithography comprising a substrate and a photo-resist layer to be formed on the substrate,
(II) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(III) a step for treating the exposed laminated resist with a developing solution,
in which said photo-resist layer comprises the polymer of claim 2.

4. A method of forming a resist pattern by immersion lithography comprising:
(Ia) a step for forming a laminated resist for immersion lithography comprising a substrate, a photo-resist layer to be formed on the substrate and a protective layer to be formed on the photo-resist layer,
(IIa) a step for immersion exposing by irradiating the laminated resist with energy rays through a photo-mask having a desired pattern and a reduction projection lens in a state of liquid being filled between the reduction projection lens and the laminated resist, thereby selectively exposing a specific region of the photo-resist layer corresponding to a photo-mask pattern, and
(IIIa) a step for treating the exposed laminated resist with a developing solution,
in which said photo-resist layer and/or protective layer comprises the polymer of claim 2.
